SEQUENCE LISTING (1 sequence)

SEQ ID NO: 1 - protein, 31 aa, synthetic construct
  source 1..31: note = PDC-E2155-185 peptide
KVGEKLSEGDLLAEIETDKATIGFEVQEEGY

(19) United States
(12) Patent Application Publication
(10) Pub. No.: US 2024/0408186 A1
(43) Pub. Date: Dec. 12, 2024

(54) TREATMENT OF PRIMARY BILIARY CHOLANGITIS (PBC) WITH TOLERIZING NANOPARTICLES

(71) Applicant: COUR PHARMACEUTICALS DEVELOPMENT COMPANY INC., Skokie, IL (US)

(72) Inventors: John J. Puisis, Skokie, IL (US); Michael Boyne, Skokie, IL (US); Greta Wodarcyk, Skokie, IL (US); Adam Elhofy, Chicago, IL (US)

(21) Appl. No.: 18/702,114

(22) PCT Filed: Oct. 21, 2022
(86) PCT No.: PCT/US22/78545

§ 371 Date: Apr. 17, 2024

Related U.S. Application Data
(60) Provisional application No. 63/369,574, filed on Jul. 27, 2022.
(60) Provisional application No. 63/270,447, filed on Oct. 21, 2021.

(51) Int. Cl. A61K 39/00; A61K 9/51; A61K 45/06; A61P 37/06

(52) U.S. Cl. CPC A61K 39/0008; A61K 9/5153; A61K 45/06; A61P 37/06; A61K 2039/545; A61K 2039/55555; A61K 2039/55566; A61K 2039/577

(57) ABSTRACT

The present disclosure relates to methods of treating Primary Biliary Cholangitis (PBC) using tolerizing immune modifying nanoparticles encapsulating PBC associated antigens.

### CROSS REFERENCE TO RELATED APPLICATIONS

This application claims the priority benefit of U.S. Provisional Patent Application No. 63/270,447, filed Oct. 21, 2021 and U.S. Provisional Patent Application No. 63/369,574, filed Jul. 27, 2022, hereby incorporated by reference in their entireties.

### FIELD OF THE DISCLOSURE

The present disclosure relates to methods of treating Primary Biliary Cholangitis (PBC) using tolerizing immune modifying nanoparticles encapsulating PBC associated antigen.

### BACKGROUND

Primary Biliary Cholangitis (PBC), also referred to as primary biliary cirrhosis, is a rare liver autoimmune disease. PBC affects women more frequently with an estimated prevalence of approximately 580 per million women in the United States¹. PBC is characterized by immune mediated destruction of intrahepatic bile duct via autoreactive T cells and B cells leading to cholestasis (reduction or stoppage of bile flow) and following a progressive course ultimately resulting in end-stage liver disease^{2,3}.

Autoimmune activity in PBC is targeted primarily to intracellular proteins (e.g., mitochondrial, and nuclear proteins) with approximately 95% patients exhibiting circulating anti-mitochondrial antibodies^{4,5}. Symptoms of PBC include toxic bile acid buildup in the liver, abnormal liver enzyme levels, liver dysfunction, liver fibrosis, cirrhosis, aching bones, fatigue, pruritis, and dry eyes and mouth.

There is no cure for PBC. The current standard of treatment for PBC relies on modulators of bile acid synthesis (e.g., ursodeoxycholic acid (UDCA))^{6,7}. Current standard of care is associated with significant side effects that fail to address the underlying immune imbalance leading to disease pathology with poor response rates. These therapeutic approaches can only delay disease progression and time to liver transplantation.

### SUMMARY

Tolerizing immune modifying particles (TIMPs), comprising one or more antigens, have been previously described for the treatment of immune-mediated disorders (e.g., autoimmune diseases and allergies) via induction of antigen-specific immune tolerance (WO2013/192532 and WO2015/023796 incorporated herein by reference). In several preclinical models of autoimmune diseases and allergies, TIMPs have demonstrated efficacy at inducing T-cell tolerance. Induction of antigen-specific tolerance to PBC autoantigens using TIMPs encapsulating PBC-specific antigens (TIMP-PBC) could ameliorate or potentially cure PBC.

Provided herein is a method of treating PBC in a subject comprising administering to the subject TIMP-PBC, wherein TIMP-PBC is administered at a dose determined based on the subject's weight. In various embodiments, TIMP-PBC is administered at a dose of 0.01 to 12 mg/kg. In various embodiments, TIMP-PBC is administered at a fixed dose of between 1 mg to 800 mg. In various embodiments, the TIMP-PBC is administered at a dose from about from about 0.01 to 12 mg/kg, from about 0.05 to 10 mg/kg, from about 0.01 to 5 mg/kg, from about 0.1 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg. In various embodiments, TIMP-PBC is administered at a dose of about 0.01 mg/kg, 0.05 mg/kg, 0.1 mg/kg, 0.25 mg/kg, 0.5 mg/kg, 1.0 mg/kg, 2.0 mg/kg, 3.0 mg/kg, 4.0 mg/kg, 5 mg/kg, 6 mg/kg, 8.0 mg/kg, 10 mg/kg, or 12 mg/kg. In various embodiments, TIMP-PBC is administered at a fixed dose of about 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

In various embodiments, TIMP-PBC encapsulates one or more PBC antigens or antigenic epitopes. In various embodiments, the PBC antigens are a mitochondrial protein. In various embodiments, the PBC antigens are nuclear proteins. In various embodiments, the PBC antigens are selected from the group consisting of pyruvate dehydrogenase complex E2 subunit (PDC-E2), gp210, nucleoporin 62, Sp100, PML, CENP A, CENP B, CENP C, dsDNA, histone, branched-chain 2-oxo-acid dehydrogenase complex (BCOADC), smooth muscle protein, soluble liver antigen, liver/kidney microsome, centromere protein, tubulin, actin, vimentin, desmin, cytokeratin, F-actin, UDP glucuronosyltransferase Family 1 Member A Complex (UGTA1), formiminotransferase cyclodeaminase, asialoglycoprotein receptor (ASGPR), cardiolipin, h-Lamp-2, proteinase 3, CYP 2C9, CYP 2A6, and CYP P450 2D6.

In various embodiments, TIMP-PBC encapsulates PDC-E2 peptide comprising an antigenic epitope. In various embodiments, TIMP-PBC encapsulates PDC-E2 amino acids 155-185 (PDC-E2₁₅₅-185), having the amino acid sequence KVGEKLSEGDLLAEIETDKATIGFEVQEEGY) (SEQ ID NO: 1).

In various embodiments, TIMP-PBC is administered in a single dose or in multiple doses. In various embodiments, TIMP-PBC is administered in two doses one-week apart. In various embodiments, TIMP-PBC is administered once weekly, once every two weeks, once every three weeks, once every 4 weeks, once every two months, once every three months, once every 6 months, or once per year.

In various embodiments, TIMP-PBC consists of poly (lactic co-glycolic acid) (PLGA) particles encapsulating one or more PBC antigens and a suitable buffering agent or excipient. In various embodiments, TIMP-PBC particles are surface functionalized. In various embodiments, TIMP-PBC particles are surface functionalized by carboxylation. In various embodiments, TIMP-PBC particles have a negative zeta potential. In various embodiments, the negative zeta potential of TIMP-PBC particles is between about −100 mV to about 0 mV. In various embodiments, the zeta potential of the particles is from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV.

In various embodiments, the size, or diameter, of TIMP-PBC particles is between 0.05 μm to about 10 μm. In various embodiments, the diameter of TIMP-PBC particles is between 0.1 μm and about 10 μm. In various embodiments, the diameter of TIMP-PBC particles is between 0.1 μm and about 5 μm. In various embodiments, the diameter of TIMP-PBC particles is between 0.1 μm and about 3 μm. In various embodiments, the diameter of TIMP-PBC particles is between 0.3 μm and about 5 μm. In various embodiments, the diameter of TIMP-PBC particles is about 0.3 μm to about 3 μm. In various embodiments, the diameter of TIMP-PBC particles is between about 0.3 μm to about 1 μm. In various embodiments, the diameter of TIMP-PBC particles is between about 0.4 μm to about 1 μm. In various embodiments, the TIMP-PBC particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PBC particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the diameter of the negatively charged particle is between 400 nm to 800 nm.

In various embodiments, TIMP-PBC is administered intravenously, subcutaneously, intramuscularly, intraperitoneally, intranasally, or orally.

In various embodiments, TIMP-PBC is administered at a concentration of between about 0.005 mg/mL and about 50 mg/mL. In various embodiments, TIMP-PBC is administered at a concentration of about 0.05 mg/mL, 0.1 mg/ml, 0.5 mg/mL, 1 mg/mL, 2 mg/ml, 3 mg/mL, 3.25 mg/ml, 3.5 mg/ml, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL. In various embodiments, TIMP-PBC is administered via intravenous infusion lasting about 1, 2, 3, 4, 5, 6, 7, or 8 hours.

In various embodiments, administering TIMP-PBC to a subject in need thereof, relieves one or more symptoms of PBC. In various embodiments, the symptoms of PBC are selected from the group consisting of liver inflammation, cirrhosis, cholestasis, liver dysfunction, liver failure, liver fibrosis, increased liver immune infiltrate, elevated bile acid levels, elevated liver enzyme levels, (ALT, ALP, AST, γ-glutamyl transpeptidase), elevated bilirubin levels, circulating anti-mitochondrial antibodies (AMAs), circulating anti-nuclear antibodies (ANAs), fatigue, itchy skin, pruritis, dry eyes and mouth, abdominal pain, splenomegaly, musculoskeletal pain, edema, fluid buildup, skin xanthomas, jaundice, hyperpigmentation, osteoporosis, high cholesterol, diarrhea, steatorrhea, hypothyroidism, and weight loss.

In various embodiments, administering TIMP-PBC to a subject in need thereof reduces the duration and severity of an inflammatory immune response to one or more PBC antigens. In various embodiments, the inflammatory immune response is a T cell response, B cell response, Th1 response, myeloid cell response, and/or an antibody response. In various embodiments, the efficacy of TIMP-PBC at relieving one or more symptoms of PBC and/or reducing the duration and severity of an inflammatory immune response to one or more PBC antigens is determined from the assay of one or more biological samples from the subject. In various embodiments, the biological samples are selected from the group consisting whole-blood, peripheral blood, peripheral blood mononuclear cells (PBMCs), serum, plasma, urine, cerebrospinal fluid (CSF), stool, a tissue biopsy, and/or a bone-marrow biopsy.

In various embodiments, administering TIMP-PBC in a subject reduces levels of activated antigen-specific T cells. In various embodiments, administering TIMP-PBC in a subject reduces levels of activated antigen-specific CD4+ and/or CD8+ T cells.

In various embodiments, administering TIMP-PBC in a subject reduces levels of T cell infiltrate in the liver. In various embodiments, treatment with TIMP-PBC decreases liver immune infiltrate by about 5%-100% or by about 2-100-fold, inclusive.

In various embodiments, administering TIMP-PBC in a subject reduces levels of anti-mitochondrial antibodies in blood. In various embodiments, treatment with TIMP-PBC decreases the levels of anti-mitochondrial antibodies by about 5%-100% or by about 2-100-fold, inclusive, relative to the subject's baseline measurement and/or relative to a healthy subject.

In various embodiments, administering TIMP-PBC in a subject reduces levels of anti-nuclear antibodies in blood. In various embodiments, treatment with TIMP-PBC decreases the levels of anti-nuclear antibodies by about 5%-100% or by about 2-100-fold, inclusive, relative to the subject's baseline measurement and/or relative to a healthy subject.

In various embodiments, administering TIMP-PBC in a subject reduces levels of IgM in blood. In various embodiments, treatment with TIMP-PBC decreases the levels of IgM by about 5%-100% or by about 2-100-fold relative to the subject's baseline measurement and/or relative to a healthy subject.

In various embodiments, administering TIMP-PBC in a subject reduces levels of anti-Gp210 antibody, anti-Sp100 antibody, kynurenine and/or soluble CD14 in blood. In various embodiments, treatment with TIMP-PBC decreases the levels of anti-Gp210 antibody, anti-Sp100 antibody, kynurenine and/or CD14 by about 5%-100% or by about 2-100-fold relative to the subject's baseline measurement and/or relative to a healthy subject.

In various embodiments, the disclosure provides a method of treating PBC or PBC related symptoms, in a subject in need thereof comprising administering to the subject a composition comprising TIMP-PBC alone or in combination with a therapeutic useful to treat PBC. In various embodiments, the therapeutic useful to treat PBC is a steroid, corticosteroid, nonsteroidal immunosuppressive agent, immunomodulatory agent, monoclonal antibody, cytokine and chemokine targeting therapy, JAK inhibitor, chimeric antigen receptor (CAR) T-cell therapy, regulatory T-cell (Treg) therapy, B-cell targeting therapy, antiviral drugs, synthetic bile acids, or surgical treatment. In various embodiments, the therapeutic is ursodeoxycholic acid (UDCA), obeticholic acid (OCA), fibrates, peroxisome proliferator-activated receptor δ (pparδ) agonist, ileal bile acid transporter (IBAT) inhibitor, ustekinumab, tauroursodeoxycholic acid, 6α-ethyl chenodeoxycholic acid, setanaxib, moexipril, abatacept, fibroblast growth factor, statin, colchicone, CD20 targeting therapy, CD19 targeting therapy, CD40/CD40L targeting therapy, CD80/CD86 targeting therapy, B cell targeting factor (BAFF) targeting therapy, B cell maturation antigen (BCMA) targeting therapy, anti-IL6 therapy, anti-IFN therapy, amifampridine, cyclosporine, cyclophosphamide, batoclimab, inebilizumab, nipocalimab, pozelimab, rituximab, satralizumab, seldelapar, pentoxifylline, tocilizumab, tofacitinib, tolebrutininb, avorstatin, fenofibrate, bezafibrate, linerixibat, methotrexate, butyrate, palmitate, or liver transplant. In various embodiments, the steroid or corticosteroid is selected from the group comprising beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, or hydrocortisone.

In various embodiments the therapeutic is administered prior to, concomitantly with or subsequent to the administration of TIMP-PBC. In various embodiments, the therapeutic is administered 1, 2, 3, 4, 5, 6, or 7 days prior to administration of TIMP-PBC. In various embodiments, the therapeutic is administered 1, 2, 3, or 4 weeks prior to administration of TIMP-PBC. In various embodiments, the therapeutic is administered 1, 2, 3, 4, 5, 6, 7, 8, 9, 10, 11, or 12 months prior to administration of TIMP-PBC. In various embodiments, the therapeutic is administered 1, 2, 3, 4, 5, 6, 7, 8, 9, 10, 11, or 12 years prior to administration of TIMP-PBC. In various embodiments, the therapeutic is administered 1, 2, 3, 4, 5, 6, or 7 days subsequent to administration of TIMP-PBC. In various embodiments, the therapeutic is administered 1, 2, 3, or 4 weeks subsequent to administration of TIMP-PBC. In various embodiments, the therapeutic is administered 1, 2, 3, 4, 5, 6, 7, 8, 9, 10, 11, or 12 months subsequent to administration of TIMP-PBC. In various embodiments, the therapeutic is administered 1, 2, 3, 4, 5, 6, 7, 8, 9, 10, 11, or 12 years subsequent to administration of TIMP-PBC.

Also contemplated is a composition comprising TIMP-PBC as described herein for use in treating primary biliary cholangitis. In various embodiments, the disclosure provides for use of a composition comprising TIMP-PBC as described herein in the preparation of a medicament for treating primary biliary cholangitis.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1. CNP-104 inhibits antigen-specific T cell proliferation in the PBC mouse model. PDC-E2 specific T cell proliferation of mice treated with CNP-104 Lot 2107GM07. The PDC-E2 specific T cell proliferation of the CNP-104 treatment group was compared to the control unloaded CNP group in the PBC mouse model. **** represents p<0.0001.

FIG. 2. CNP-104 inhibits anti-mitochondrial IgG antibody in PBC mouse model. PDC-E2 specific IgG anti-mitochondrial antibody (AMA) titers were determined from mice primed with PDC-E2₁₅₅-185 and treated with CNP-104 or control unloaded nanoparticles (CNP) by ELISA.

FIG. 3A-3B. CNP-104 inhibits antigen-specific T cell response in the DTH model. C57BL/6 mice were primed with PDC-E2-Peptide emulsified in CFA on Day 0. On Day 0 and Day 7 post priming, mice were treated with CNP 104 at a dose of 2.5 mg dose/mouse (10 mg/kg HED). On Day 14 post priming, the mice were challenged intradermally with PDC-E2-Peptide (single lot, FIG. 3A; multiple lots, FIG. 3B) or Ovalbumin (negative control). The pinna thickness of each ear was measured immediately post-elicitation with PDC-E2-Peptide and OVA as well as 24 hours post elicitation. The change in pinna thickness (ΔT) for both ears of each mouse was calculated to assess the DTH response.

FIG. 4. CNP-104 inhibits leukocyte infiltration in liver in the PBC mouse model. Levels of leukocyte (CD45+) infiltration in liver were determined from mice primed with PDC-E2155-185 by flow cytometry.

FIG. 5. CNP-104 increases regulatory T-cells in the liver. On the day of experiment initiation (Day 0), mice were primed with 2-octynoic acid conjugated to bovine serum albumin (2OA-BSA)/CFA by intraperitoneal injection (i.p.). 2OA-BSA i.p. priming was repeated with IFA on Day 14. On Day 0 and Day 2 mice were injected i.p. with 100 ng of pertussis toxin. On Day 0 and Day 14 mice were injected subcutaneously with 5 mg/kg poly(I:C). On Day 7 mice were primed with PDC-E2-Peptide/CFA emulsion. Mice were treated intravenously with CNP 104 on day 7 and day 14. Livers were analyzed for Tregs on day 55. CNP 104 treatment increased the frequency of Tregs in the liver compared to naive untreated control.

FIGS. 6A and 6B. Schedule of events for the clinical trial evaluating safety and efficacy of CNP-104.

FIG. 7. Illustrates HLA haplotype, presence of anti-mitochondrial antibodies (AMA) and levels of alkaline phosphatase in blood of PBC patients 70% of PBC patients express HLA haplotype restriction for PDC-E2. 70% and 76% of PBC patients are positive for AMA and have elevated ALP levels, respectively.

FIG. 8 shows cell counts for CD4+ T cells, CD8+ T cells and biliary epithelial cells evaluated from liver biopsies using a multiplex immune-fluorescence panel.

### DETAILED DESCRIPTION

There is a need for therapeutics for addressing immune imbalance in PBC leading to improved disease symptoms and improved outcomes without the risk of toxic side-effects. The present disclosure provides methodology for monitoring the induction of and maintenance of immunologic tolerance in a subject having PBC after receiving immunotherapy.

The headings herein are for the convenience of the reader and not intended to be limiting. Additional aspects, embodiments, and variations of the invention will be apparent from the Detailed Description and/or Drawing and/or claims.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “immune modifying particle”, the term “carrier particle”, and the term “bead” may be used interchangeably depending on the context. Additionally, the term “particle” may be used to encompass beads and spheres.

“Negatively charged particle” as used herein refers to particles which have been modified to possess a net surface charge that is less than zero.

“Carboxylated particles” or “carboxylated beads” or “carboxylated spheres” includes any particle that has been modified to contain a carboxyl group on its surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups.

As used herein, the term “Th cell” or “helper T cell” refers to CD4⁺ cells. CD4⁺ T cells assist other white blood cells with immunologic processes, including maturation of B cells into plasma cells and memory B cells, and activation of cytotoxic T cells and macrophages. T cells become activated when they are presented with peptide antigens by MHC class II molecules, which are expressed on the surface of antigen-presenting cells (APCs).

As used herein, the term “Th1 cell” refers to a subset of Th cells which produce proinflammatory mediators. Th1 cells secrete cytokines to facilitate immune response and play a role in host defense against pathogens in part by mediating the recruitment of neutrophils and macrophages to infected tissues. Th1 cells secrete cytokines including IFN-gamma, IL2, IL-10, and TNF alpha/beta to coordinate defense against intracellular pathogens such as viruses and some bacteria.

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include post-expression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally-occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising via host cells that produce the proteins or through errors due to PCR amplification prior to host cell transfection.

“Antigenic moiety” or “antigen” as used herein refers to any moiety, for example a peptide, that is recognized by the host's immune system. Examples of antigenic moieties include, but are not limited to, autoantigens, allergens, enzymes, and/or bacterial or viral proteins, peptides, drugs or components.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three-dimensional structure and/or amino acid sequence that is identical or is similar to the epitope used to generate the antibody, yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure and/or the amino acid sequence of the epitope and mimotope.

The term “symptom” is used herein to mean any physical or observable manifestation of a disorder, whether it is generally characteristic of that disorder or not. The term “symptoms” can mean all such manifestations or any subset thereof.

The term “therapeutically effective amount” is used herein to indicate the amount of antigen-specific composition of the disclosure that is effective to ameliorate or lessen symptoms or signs of disease to be treated.

The terms “treat”, “treated”, “treating” and “treatment”, as used with respect to methods herein refer to eliminating, reducing, suppressing or ameliorating, either temporarily or permanently, either partially or completely, one or more clinical symptom, manifestation or progression of an event, disease or condition. Such treating need not be absolute to be useful. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### Particles

The size and charge of the particles are important for tolerance induction. While the particles will differ in size and charge based on the antigen encapsulated within them, in general, particles described herein are effective at inducing tolerance when they are between about 100 nanometers and about 1500 nanometers and have a negative charge of between 0 to about −100 mV. In various embodiments, the particles are 400-800 nanometers in diameter and have a charge of between about −25 mV and −70 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PGA particles, PLA particles, PLGA particles, PLURIONICS stabilized polypropylene sulfide particles, and diamond particles.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS® (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles disclosed herein may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22:1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly (lactic-co-glycolic) acid copolymers (PLGA or PLG; the terms are interchangeable), poly (lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

In certain embodiments, the particle is a co-polymer having a molar ratio from about 80:20 to about 100:0, or about 20:80 to 100:0. Suitable co-polymer ratio of present tolerizing immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In certain embodiments, the particle is a PLURONICS stabilized polypropylene sulfide particle, a polyglycolic acid particle (PGA), a polylactic acid particle (PLA), or a poly (lactic-co-glycolic acid) particle, or a carboxlated polyglycolic acid particle (PGA), carboxylated polylactic acid particle (PLA), or carboxylated poly (lactic-co-glycolic acid) particle. In certain embodiments, the particle has a copolymer ratio of polylactic acid/polyglycolic acid 80:20:polylactic acid/polyglycolic acid 90:10:polylactic acid/polyglycolic acid 50:50. In various embodiments, the particle is a poly (lactic-co-glycolic acid) particle and has a copolymer ratio of about 50:50 polylactic acid:polyglycolic acid.

It is contemplated that the particle may further comprise a surfactant. The surfactant can be anionic, cationic, or nonionic. Surfactants in the poloxamer and poloaxamines family are commonly used in particle synthesis. Surfactants that may be used, include, but are not limited to PEG, Tween-80, gelatin, dextran, pluronic L-63, poly vinyl alcohol (PVA), poly acrylic acid (PAA), methylcellulose, lecithin, dimethylaminobenzaldehyde (DMAB) and poly(ethyl methacrylate) (PEMA). Additionally, biodegradable and biocompatible surfactants including, but not limited to, vitamin E TPGS (D-a-tocopheryl polyethylene glycol 1000 succinate), poly amino acids (e.g., polymers of lysine, arginine, aspartic acid, glutamic acid, serine, threonine, tyrosine and cysteine, or their enantiomers), and sulfate polymers. In certain embodiments, two surfactants are used. For example, if the particle is produced by a double emulsion method, the two surfactants can include a hydrophobic surfactant for the first emulsion, and a hydrophobic surfactant for the second emulsion. In certain embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to reduce the nanoparticle size. One strategy to accomplish this is to apply a second strong shear rate. The leakage effect can be reduced by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference.

### Antigens

An antigen refers to a discreet portion of a molecule, such as a polypeptide or peptide sequence, a 3-dimensional structural formation of a polypeptide or peptide, a polysaccharide or polynucleotide that can be recognized by a host immune cells. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity or re-programming of T cells to T cell receptor-mediated stimulation. Such re-programming is generally antigen-specific and persists after exposure to the antigenic peptide has ceased. This reprogramming leads to induction of regulatory T cells, Tr1 cells and T cell anergy. For example, insensitivity in T cells is characterized by lack of effector cytokine production, lack of proliferation, or lack of activation. Re-programming occurs when T cells are exposed to antigen and receive a first signal (a T cell receptor or CD3 mediated signal) in the absence of a second signal (a costimulatory signal) or presence of an inhibitory signal (negative costimulation or regulatory cytokines). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with (e.g., IL-2).

It is contemplated that the tolerizing therapy described herein is antigen-specific. For example, TIMPs administered as tolerizing therapy encapsulate one or more antigens associated with said tolerizing therapy and associated disease or condition being treated. It is contemplated that the TIMPs used in tolerizing therapy comprise one or more PBC antigens. The one or more PBC antigens may be whole proteins, polypeptides, or peptides comprising PBC antigenic epitopes.

In certain embodiments, one, two, three, or a higher number of antigens or antigenic peptides are used in the TIMPs. In certain embodiments, the one or more PBC antigens is encapsulated in the TIMP by covalent linkage to the interior surface of the particle (See e.g., US Patent Publication US20190282707, herein incorporated by reference). In certain embodiments, it is contemplated that sequences of two or more PBC antigens are linked in a fusion protein and encapsulated within a TIMP described herein. Certain PBC epitopes are described in Shimoda et al. (Gastroenterology 124:1915-1925, 2003). Methods for making TIMP with linked epitopes are described in US Patent Publication US20190365656, herein incorporated by reference.

### Methods of Use

Primary biliary cholangitis (PBC, previously referred to as primary biliary cirrhosis) is a prototypical autoimmune liver disease characterized by destructive lymphocytic cholangitis and specific anti-mitochondrial autoantibodies (AMAs) targeted primarily at the E2 component of the mitochondrial pyruvate dehydrogenase complex (PDC-E2).¹ The prevalence of PBC in the United States is estimated at approximately 580 per million women.² There is a significant unmet medical need with this rare disease and an opportunity where TIMP-PBC will positively impact subjects suffering from this disease.

The most common cause of death in patients with PBC is liver failure.² Current standard of care treatment for PBC (ursodeoxycholic acid) has reduced the likelihood of needing a liver transplant and has increased patient life span. However, up to 40% of patients with PBC have a suboptimal biochemical response to treatment as determined either by binary response criteria and/or prognostic models.¹

Treatments currently in clinical trials for PBC fall into multiple categories: synthetic bile acids, bile acid inhibitors, Peroxisome proliferator-activated receptor (PPAR) gamma agonists, and immune suppressants such as rituximab and abetacept. However, these classes of therapies do not address the underlying cause of liver pathology that is driven by antigen specific autoimmune attack on the liver, both directly by CD8 T cells and indirectly by antibodies.

Provided herein is a method of treating PBC in a subject comprising administering to the subject TIMP-PBC, wherein TIMP-PBC is administered at a dose level determined based on the subject's weight. It is also contemplated that TIMP-PBC may be administered at a fixed dosage irrespective of the subject's weight. In various embodiments, contemplated is a method of treating PBC in a subject comprising administering to the subject TIMP-PBC, wherein TIMP-PBC is administered at a dose of 0.01 to 12 mg/kg based on the subject's weight or at a fixed dose between 1 mg and 800 mg. Also provided herein is a method of reducing an inflammatory immune response to PBC antigens in a subject suffering from PBC comprising administering to the subject TIMP-PBC, wherein TIMP-PBC is administered at a dose of 0.01 to 12 mg/kg based on the subject's weight or at a fixed dose between 1 mg and 800 mg.

Also contemplated, the TIMP-PBC is administered at a dose from about 0.01 to 12 mg/kg, from about 0.05 to 10 mg/kg, from about 0.01 to about 5 mg/kg, from about 0.1 to about 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg. Optionally, the TIMP-PBC is administered in a dose of about 0.01, mg/kg, 0.02 mg/kg, 0.05 mg/kg, 0.1 mg/kg, 0.25 mg/kg, 0.5 mg/kg, 1.0 mg/kg, 2.0 mg/kg, 4.0 mg/kg, 6 mg/kg, 8.0 mg/kg, 10 mg/kg, or 12 mg/kg. Alternatively, TIMP-PBC is administered at a dose of about 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In another embodiment, TIMP-PBC is administered at a concentration of between about 0.005 mg/ml and about 50 mg/mL, optionally about 0.05 mg/mL, 0.1 mg/mL, 0.5 mg/mL, 1 mg/mL, 2 mg/ml, 3 mg/mL, 3.25 mg/mL, 3.5 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

It is contemplated that the TIMP-PBC is administered in a single dose or in multiple doses. In various embodiments, TIMP-PBC is administered once weekly, once every two weeks, once every three weeks, once every 4 weeks, once every two months, once every three months, once every 6 months, once per year, once every two years, once every three years, once every four years, once every five years, once every six years, once every seven years, once every eight years, once every nine years, or once every ten years. In certain embodiments, TIMP-PBC is administered in two doses one-week apart.

In various embodiments, TIMP-PBC is administered intravenously, subcutaneously, intramuscularly, intraperitoneally, intranasally, or orally. It is contemplated that if TIMP-PBC is given intravenously, it can be via intravenous infusion lasting about 1, 2, 3, 4, 5, 6, 7, or 8 hours.

TIMP-PBC therapy is contemplated to relieve, lessen or ameliorate one or more symptoms of PBC. Symptoms of PBC include, but are not limited to, liver inflammation, cirrhosis, cholestasis, liver dysfunction, liver failure, liver fibrosis, increased liver immune infiltrate, elevated bile acid levels, elevated liver enzyme levels, (ALT, ALP, AST, γ-glutamyl transpeptidase), elevated bilirubin levels, circulating anti-mitochondrial antibodies (AMAs), circulating anti-nuclear antibodies (ANAs), fatigue, itchy skin, pruritis, dry eyes and mouth, abdominal pain, splenomegaly, musculoskeletal pain, edema, fluid buildup, skin xanthomas, jaundice, hyperpigmentation, osteoporosis, high cholesterol, diarrhea, steatorrhea, hypothyroidism, and weight loss.

TIMP-PBC therapy is also contemplated to reduce, shorten or ameliorate the duration and severity of an inflammatory immune response to one or more PBC antigens in a subject. An inflammatory immune response includes a T cell response, B cell response, Th1 response, myeloid cell response, and/or an antibody response. In various embodiments, the efficacy of TIMP-PBC at relieving one or more symptoms of PBC and/or reducing the duration and severity of an inflammatory immune response to one or more PBC antigens is determined from the assay of one or more biological samples from the subject as described herein.

### Screening Methods

It is contemplated that induction of, and maintenance of immunological tolerance is monitored in a subject suffering from PBC, wherein the subject is treated, or about to undergo treatment, with antigen-specific tolerizing therapy comprising TIMPs encapsulating PBC antigens as described herein.

Methods of screening for cell types, cytokines or other measures of tolerance from a subject undergoing tolerizing therapy as described herein are known in the art. Methods of assessing tolerance are done using such techniques as flow cytometry, Mass Cytometry (CyTOF), ELISA, ELISPOT, in vitro or ex vivo cell stimulation assays (including, but not limited to, cell proliferation assays, macrophage stimulation assays), measuring autoantibodies or measuring immunoblobulin (Ig) serotype, e.g., by ImmunoCap assay.

In various embodiments, the immune tolerance status of a subject is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, enzymes, and/or combinations thereof relevant in the disease or disorder.

Cells assayed from the biological sample include immune cells, non-immune cells, and/or combinations thereof. Immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

One aspect of a subject's immune tolerance status, and immune signature, is determined by analyzing one or more proteins from one or more biological sample(s) from the subject. In various embodiments, the proteins are cytokines and/or chemokines. In various embodiments the proteins are cell signaling proteins. In various embodiments, the cytokines and chemokines are selected from the group consisting of IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, soluble CD14, and/or combinations thereof. In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, and EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. Several methods for the detection of immunoglobulins from a biological sample have been described in the literature including ELISA and ImmunoCap.

One aspect of a subject's immune tolerance status, and immune signature, is determined by analyzing one or more cell-surface proteins from a biological sample(s). In various embodiments, the cell-surface proteins include CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2Rβ, IL-4R, IL-5Rα, CSF2Rβ, IL-6Rα, gp130, IL-7Rα, IL-9R, IL-10R, IL-12Rβ1, IL-12Rβ2, IL-13Rα1, IL-13Rα2, IL-15Rα, IL-21R, IL23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α, β, γ, δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In various embodiments, a subject's immune tolerance status, and immune signature, is determined by analyzing the subject's liver function. In various embodiments, the subject's liver function is determined by the assay of total cholesterol, triglyceride, LDL-cholesterol, aspartate aminotransferase (AST), alanine aminotransferase (ALT), gamma glutamyl transferase (GGT), alkaline phosphatase (ALP), albumin, total protein, total bilirubin, globulin, creatine kinase (CK), and lactate dehydrogenase (LDH).

One aspect of a subject's immune tolerance status, and immune signature, is determined by analyzing one or more metabolites from the biological sample(s). In various embodiments, the metabolite is an inflammatory metabolite. In various embodiments, the metabolite is an anti-inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), kynurenine, 3-hydroxy kynurenine, lipoxin A4, and lipoxin B4. In various embodiments, examples of anti-inflammatory metabolites include 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine.

A list of human metabolites that can be assayed from a biological sample can be found in the literature including in (Psychogios et al., 2011), (Wishart et al., HMDB: the Human Metabolome Database. Nucleic Acids Res. 2007 January; 35 (Database issue): D521-6, 2007), and the Human Metabalome Database (HMDB) and are incorporated herein by reference.

In certain embodiments, the subject's tolerance status is determined by analyzing nucleic acids from the biological sample(s). In various embodiments, the nucleic acids are DNA and/or RNA, including, but not limited to, single stranded DNA, double stranded DNA, mRNA, rRNA, tRNA, siRNA, miRNA, long non-coding RNAs (long ncRNAs, lncRNA), non-coding RNA (ncRNA), and mitochondrial RNA. In various embodiments, the subject's immune tolerance status is determined by assaying gene expression from the biological sample(s). In various embodiments, the immune tolerance status is determined by assaying gene expression associated with immune function, an antibody, foreign body response, metabolism, apoptosis, cell death, necrosis, ferroptosis, autophagy, cell migration, endocytosis, phagocytosis, pinocytosis, tight-junction regulation, cell adhesion, differentiation, and/or combinations thereof. In various embodiments, the immune tolerance status is determined by assaying gene expression associated with immune suppression. In various embodiments, the immune tolerance status is determined by assaying gene expression associated with immune activation. In various embodiments, the immune tolerance status is determined by assaying gene expression associated with immune regulatory functions. In various embodiments, nucleic acid analysis is used to generate an immune tolerance signature. Several methodologies have been described in the literature for high-throughput gene expression analysis including RNA sequencing (RNA-seq), single-cell RNA sequencing (scRNA-seq), exome sequencing, and microarray-based analyses.

The biological sample is optionally assayed after in vivo and/or ex vivo stimulation with one or more stimuli such as an antigen and one or more activating agents. It is contemplated that the T cells, B cells, and immunoglobulins used in the assay are antigen specific. Exemplary T cells include effector memory T cells, antigen-specific T cells, activated antigen-specific T cells, Th1 cells, Th17 cells, T follicular helper (TFH) cells, THO cells, or other antigen-specific T cells. B cells include effector B cells, memory B cells, plasma cells, and Breg cells. In certain embodiments, T cells are identified based on the expression of proteins described in Table 1.

TABLE 1

| Cell Type | Expression Marker |
|---|---|
| Effector memory (EM) | CD45RA^{LO} CD4⁺ |
| T cell | |
| Disease specific T cell | CD45RA^{LO} CD4⁺ CD154⁺ CD137⁺ |
| Activated disease | CD45RA^{LO} CD4⁺ CD154⁺ CD137⁺ CD38⁺ |
| specific T cell | |
| Th2 cell | CD45RA^{LO} CD4⁺ CD154⁺ CD137⁺ CXCR4 |
| Th1 cell | CXCR5⁻ CRTH2⁻ CXCR3⁺ CCR6⁻ |
| Th17 cell | CXCR5⁻ CRTH2⁻ CXCR3⁻ CCR6⁺ |
| TFH cell | CD45RA^{LO} CD4+ CXCR5+ |
| Treg | CD154⁻ CD127⁻ CD25⁺ TIGIT⁺ |
| | CD137⁺ GARP^{+/-} OX40^{+/-} |
| Th0 cell | CXCR5⁻ CRTH2⁻ CXCR3⁻ CCR6⁻ CCR4⁻ |
| Disease-specific CD8 | CD8⁺ CD137⁺ CD69⁺ CD355⁺ |
| T cells | |
| Activated antigen | CD8⁺ CD137⁺ CD69⁺ CD355⁺ CD38⁺ |
| specific CD8 T cell | |
| Exhausted T cells | CD4⁺ TIGIT⁺ KLRG1⁺ PD-1⁺ |

In various embodiments, the immune tolerance status of the subject is determined by obtaining one or more samples, e.g., whole blood, from the subject pre-dose on the day of the first TIMP-PBC administration (Day 1), 14 days after administration of the second dose, and then at every 90 days post-second dose (e.g., Days 90, 180, 270, and 360 post-second dose). Whole blood can then be processed to isolate peripheral blood mononuclear cells (PBMCs), basophils, neutrophils, plasma, and serum for downstream analyses. Assay of cells isolated from one or more samples collected from the subject and analyzed using such methods as described below.

In various embodiments, the immune tolerance status of the subject determined prior to administration of TIMP-PBC serves as the baseline. In various embodiments, the subject's baseline is determined from the assay of one or more biological samples 1, 2, 3, 4, 5, 6, or 7 days prior to administration of TIMP-PBC. In various embodiments, the subject's baseline is determined from the assay of one or more biological samples 1, 2, 3, or 4 weeks prior to administration of TIMP-PBC. In various embodiments, the subject's baseline is determined from the assay of one or more biological samples 1, 2, 3, 4, 5, 6, 7, 8, 9, 10, 11, or 12 months prior to administration of TIMP-PBC.

In various embodiments, the immune tolerance status of the subject is determined after administration of TIMP-PBC. In various embodiments, the immune tolerance status of the subject is determined from the assay of one or more biological samples 1, 2, 3, 4, 5, 6, or 7 days after to administration of TIMP-PBC. In various embodiments, the subject's Baseline is determined from the assay of one or more biological samples 1, 2, 3, or 4 weeks after to administration of TIMP-PBC. In various embodiments, the subject's baseline is determined from the assay of one or more biological samples 1, 2, 3, 4, 5, 6, 7, 8, 9, 10, 11, or 12 months after to administration of TIMP-PBC. In various embodiments, the immune tolerance status of the subject determined after administration of TIMP-PBC is compared to the baseline. In various embodiments, the immune tolerance status of the subject determined after the administration of TIMP-PBC is compared to a healthy subject.

In various embodiments, the immune tolerance status of the subject is determined based on the assessment of liver fibrosis. In various embodiments, liver fibrosis is assessed by imaging. In various embodiments, liver fibrosis is assessed by FibroScan, ultrasonography, computed tomography (CT), magnetic resonance imaging (MRI), magnetic resonance elastography (MRA), ultrasound elastography, transient elastography, point shear wave elastography, or two-dimensional shear wave elastography. In various embodiments, liver fibrosis is assessed based on one or more biological markers. In various embodiments, the biochemical markers is from whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, biological markers are identified from the assay of cells, proteins, peptides, enzymes, metabolites, cells, and/or one or more biochemicals. In various embodiments, the proteins are cytokines, chemokines, proteases, cell surface proteins, soluble proteins, and/or antibodies. In various embodiments, the cells are immune cells or non-immune cells. In various embodiments, immune cells are T cells, B cells, NK cells, NK T cells, monocytes, macrophages, dendritic cells, eosinophils, or basophils. In various embodiments, the cells are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), red blood cells, platelets, and/or Kupffer cells. In various embodiments, the biochemical markers are total cholesterol, triglyceride, LDL-cholesterol, aspartate aminotransferase (AST), alanine aminotransferase (ALT), gamma glutamyl transferase (GGT), alkaline phosphatase (ALP), albumin, total protein, total bilirubin, globulin, creatine kinase (CK), and lactate dehydrogenase (LDH), hyaluronic acid, PIINP, TIMP-1, Type 4 collagen 7S, ProC3, or M2BPGi. In various embodiments, the metabolites are acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), kynurenine, 3-hydroxy kynurenine, lipoxin A4, lipoxin B4, 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine.

The immune tolerance signature of a subject is generated using one or more of the following parameters assayed from one or more biological samples obtained from the subject and stimulated in vivo and/or ex vivo:
A. proportion of effector T cells in the total T cell population,
B. proportion of Treg cells in the total T cell population,
C. proportion of effector B cells in the total B cell population,
D. levels of specific IgG, and/or IgM,
E. levels of inflammatory cytokines and chemokines,
F. levels of anti-inflammatory cytokines and chemokines,
G. levels of liver enzymes,
H. levels of inflammatory metabolites, and
I. levels of anti-inflammatory metabolites.

The immune tolerance signature is indicative of maintenance of immune tolerance if 1, 2, 3, 4, 5, 6, 7, 8, or 9 parameters listed in (a)-(i) above indicate maintenance of immune tolerance. In various embodiments, the immune tolerance signature is indicative of maintenance of immune tolerance if at least 2/9 parameters listed in (a)-(i) indicate maintenance of immune tolerance. In various embodiments, the subject is determined to not require treatment with TIMPs if 1, 2, 3, 4, 5, 6, 7, 8, or 9 parameters listed in (a)-(i) above indicate maintenance of immune tolerance. In various embodiments, the subject is determined to not require treatment with TIMPs if at least 3/9 parameters listed in (a)-(i) above indicate maintenance of immune tolerance.

The immune tolerance signature of a subject generated using one or more parameters described herein indicates weakening and/or absence of immune tolerance prior to or after treatment with TIMP-PBC, if:
a. the proportion of effector T cells in the total T cell population is between 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), relative to the subject's baseline measurement and/or relative to a healthy subject and/or
b. the proportion of Treg cells in the total T cell population is between 0.01-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values) relative to the subject's baseline measurement and/or relative to a healthy subject, and/or
c. the proportion of effector B cells in the total B cell population is between 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), relative to the subject's baseline measurement and/or relative to a healthy subject and/or
d. the levels of IgG and/or IgM are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
e. levels of inflammatory cytokines/chemokines are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
f. levels of anti-inflammatory cytokines and chemokines are decreased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a relative to the subject's baseline measurement and/or relative to a healthy subject, and/or
g. levels of liver enzymes are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
h. levels of inflammatory metabolites are increased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject, and/or
i. levels of anti-inflammatory metabolites are decreased by about 0.01%-100% (e.g., about 0.01%, about 0.05%, about 0.1%, about 0.2%, about 0.5%, about 1%, about 1.5%, about 2%, about 2.5%, about 3%, about 3.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

The efficacy of TIMP-PBC at relieving one or more symptoms of PBC and/or reducing the duration and severity of an inflammatory immune response to PBC antigens is determined from the assay of one or more biological samples from the subject. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, enzymes, and/or combinations thereof.

In various embodiments, the efficacy of TIMP-PBC at relieving one or more symptoms of PBC and/or reducing the duration and severity of an inflammatory immune response to PBC antigens is determined based on the assay of cells from one or more biological samples from the subject pre- and post-treatment with TIMP-PBC. In various embodiments, the cells are immune cells, non-immune cells, and/or combinations thereof. In various embodiments, immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

In various embodiments, the efficacy of TIMP-PBC at relieving one or more symptoms of PBC and/or reducing the duration and severity of an inflammatory immune response to PBC antigens is determined based on the assay of cell surface proteins from one or more biological samples from the subject pre- and post-treatment with TIMP-PBC. In various embodiments, the cell surface proteins are selected from the group consisting CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2Rβ, IL-4R, IL-5Rα, CSF2Rβ, IL-6Rα, gp130, IL-7Rα, IL-9R, IL-10R, IL-12Rβ1, IL-12Rβ2, IL-13Rα1, IL-13Rα2, IL-15Rα, IL-21R, IL23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α,β, γ, δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In various embodiments, treatment with TIMP-PBC decreases the expression of inflammatory cell surface proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject. In various embodiments, the cell surface protein is CD14.

In various embodiments, treatment with TIMP-PBC increases the expression of anti-inflammatory cell surface proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-PBC at relieving one or more symptoms of PBC and/or reducing the duration and severity of an inflammatory immune response to PBC antigens is determined based on the assay of proteins from one or more biological samples from the subject pre- and post-treatment with TIMP-PBC. In various embodiments, the proteins are cytokines and/or chemokines. In various embodiments the proteins are cell signaling proteins. In various embodiments, the cytokines and chemokines are selected from the group consisting of IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, and/or combinations thereof.

In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, caspase 1, caspase 2, caspase 3, caspase 4, caspase 5, caspase 6, caspase 7, caspase 8, Caspase 9, caspase 10, caspase 11, caspase 12, caspase 13, caspase 14, BCL-2, BCL-XL, MCL-1, CED-9, A1, BFL1, BAX, BAK, DIVA, BCL-XS, BIK, BIM, BAD, BID, and EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. Several methods for the detection of immunoglobulins from a biological sample have been described in the literature including ELISA and ImmunoCap.

In various embodiments, treatment with TIMP-PBC decreases the levels of inflammatory proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject. In various embodiments, treatment with TIMP-PBC increases the levels of anti-inflammatory proteins by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, treatment with TIMP-PBC decreases the levels of anti-nuclear antibodies (ANAs), anti-Gp210 antibody, anti-Sp100 antibody, IgM, or anti-mitochondrial antibody by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values) relative to the subject's baseline measurement and/or relative to a healthy subject.

In various embodiments, the efficacy of TIMP-PBC at relieving one or more symptoms of PBC and/or reducing the duration and severity of an inflammatory immune response to PBC antigens is determined based on the assay of metabolites from one or more biological samples from the subject pre- and post-treatment with TIMP-PBC. In various embodiments, the metabolite is an inflammatory metabolite. In various embodiments, the metabolite is an anti-inflammatory metabolite. In various embodiments, examples of inflammatory metabolites include acids, lipids, sugars, amino acids, lactate, trimethylamine N-oxide, O-acetyl creatine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), kynurenine, 3-hydroxy kynurenine, lipoxin A4, and lipoxin B4. In various embodiments, examples of anti-inflammatory metabolites include 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine. In various embodiments, treatment with TIMP-PBC decreases the levels of inflammatory metabolites by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject. In various embodiments, treatment with TIMP-PBC increases the levels of anti-inflammatory metabolites by 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-PBC at relieving one or more symptoms of PBC and/or reducing the duration and severity of an inflammatory immune response to PBC antigens is determined based on the assay of liver fibrosis from one or more liver biopsies. In various embodiments, the efficacy of TIMP-PBC at relieving one or more symptoms of PBC and/or reducing the duration and severity of an inflammatory immune response to PBC antigens is determined based on the assessment of liver fibrosis by imaging. In various embodiments, liver fibrosis is assessed by FibroScan, ultrasonography, computed tomography (CT), magnetic resonance imaging (MRI), magnetic resonance elastography (MRA), ultrasound elastography, transient elastography, point shear wave elastography, or two-dimensional shear wave elastography.

In various embodiments, the efficacy of TIMP-PBC at relieving one or more symptoms of PBC and/or reducing the duration and severity of an inflammatory immune response to PBC antigens is determined based on the assay of levels of liver immune infiltrate from one or more liver biopsies.

In various embodiments, the liver biopsy is a core liver biopsy. In various embodiments, treatment with TIMP-PBC decreases liver fibrosis by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, treatment with TIMP-PBC decreases liver immune infiltrate by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the subject's baseline measurement and/or relative to a healthy subject.

In various embodiments, the efficacy of TIMP-PBC at relieving one or more symptoms of PBC and/or reducing the duration and severity of an inflammatory immune response to PBC antigens is determined based on the subject's score on a clinical disease scale. In various embodiments, the clinical disease scale is the PBC-40 scale, Modified PBC-40 scale, Enhanced Liver Fibrosis (ELS) score, PBC-27 scale, GLOBE scale, UK-PBC scale, Mean Worst Daily Itch scale, 5-D itch scale, Visual Analogue Scale (VAS), Scheuer Scale, Nakanuma Stage, Fibrosis Score, Bile Duct Loss Score, FIB-4 Index, or APRI. A number of clinical disease scoring systems have been described in the literature and are incorporated herein by reference⁸⁻¹⁰.

In various embodiments, the efficacy of TIMP-PBC at relieving one or more symptoms of PBC and/or reducing the duration and severity of an inflammatory immune response to PBC antigens is determined based on the following assessments pre- and post-TIMP-PBC administration:
A. Levels of T cell infiltrate from a core liver biopsy.
B. Levels of antigen-specific IFN-γ production in blood.
C. Levels of serum ALP.
D. Levels of serum bilirubin.
E. Levels of serum ALT.
F. Levels of serum AST.
G. Levels of serum GGT.
H. Levels of anti-Gp210 antibody in blood.
I. Levels of anti-Sp100 antibody in blood.
J. Levels of serum soluble CD14.
K. Levels of serum kynurenine.
L. Levels of serum IgM.
M. Enhanced liver fibrosis (ELF) score.
N. Levels of liver inflammation and fibrosis in liver core biopsy.
O. Mean Worst Daily Itch Score.
P. PBC-40 scale score.

The efficacy of TIMP-PBC at improving one or more symptoms of PBC is determined from one or more of the following parameters assayed from one or more biological samples obtained from the subject:
A. proportion of effector T cells in the total T cell population,
B. proportion of Treg cells in the total T cell population,
C. proportion of effector B cells in the total B cell population,
D. levels of specific IgG, and/or IgM,
E. levels of inflammatory cytokines and chemokines,
F. levels of anti-inflammatory cytokines and chemokines,
G. levels of liver enzymes,
H. levels of inflammatory metabolites, and
I. levels of anti-inflammatory metabolites.

In various embodiments, the efficacy of TIMP-PBC at improving one or more symptoms of PBC is determined from the result of the assay of 1, 2, 3, 4, 5, 6, 7, 8, or 9 parameters listed in (a)-(i) above.

In various embodiments, efficacy of TIMP-PBC at improving one or more symptoms of PBC is identified based on HLA haplotype, presence/levels of anti-mitochondrial antibodies and levels of alkaline phosphatase in blood.

In various embodiments, efficacy of TIMP-PBC at improving one or more symptom of PBC and/or reducing the duration and severity of an inflammatory response to PBC antigens is determined based on the use of alternative PBC therapies. In various embodiments, administration of TIMP-PBC decreases the use of alternative PBC therapies. In various embodiments, the therapy is a steroid, corticosteroid, nonsteroidal immunosuppressive agent, immunomodulatory agent, monoclonal antibody, cytokine and chemokine targeting therapy, JAK inhibitor, chimeric antigen receptor (CAR) T-cell therapy, regulatory T-cell (Treg) therapy, B-cell targeting therapy, antiviral drugs, or surgical treatment.

In various embodiments, the therapy is ursodeoxycholic acid (UDCA), obeticholic acid (OCA), fibrates, peroxisome proliferator-activated receptor δ (pparδ) agonist, ileal bile acid transporter (IBAT) inhibitor, tauroursodeoxycholic acid, 6α-ethyl chenodeoxycholic acid, setanaxib, moexipril, abatacept, fibroblast growth factor, statin, colchicone, ustekinumab, CD20 targeting therapy, CD19 targeting therapy, CD40/CD40L targeting therapy, CD80/CD86 targeting therapy, B cell targeting factor (BAFF) targeting therapy, B cell maturation antigen (BCMA) targeting therapy, cytokine/chemokine inhibitor, anti-IL6 therapy, anti-IFN therapy, amifampridine, cyclosporine, cyclophosphamide, batoclimab, inebilizumab, nipocalimab, pozelimab, rituximab, satralizumab, seldelapar, pentoxifylline, tocilizumab, tofacitinib, tolebrutininb, avorstatin, fenofibrate, bezafibrate, linerixibat, methotrexate, or liver transplant.

In various embodiments, the steroid or corticosteroid is selected from the group comprising beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, or hydrocortisone.

In various embodiments, administration of TIMP-PBC decreases use of alternative PBC therapies 1%-100% (e.g. about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% relative to the subject's baseline measurement and/or relative to a healthy subject. In various embodiments, the use of alternative PBC therapies is reduced or eliminated 1, 2, 3, 4, 5, 6, or 7 days after administration of TIMP-PBC. In various embodiments, the use of alternative PBC therapies is reduced or eliminated 1, 2, 3, or 4 weeks after administration of TIMP-PBC. In various embodiments, the use of alternative PBC therapies is reduced or eliminated 1, 2, 3, 4, 5, 6, 7, 8, 9, 10, 11, or 12 months after administration of TIMP-PBC. In various embodiments, the use of alternative PBC therapies is reduced or eliminated 1, 2, 3, 4, 5, 6, 7, 8, 9, 10, 11, or 12 years after administration of TIMP-PBC.

Cells assayed from the biological sample include immune cells, non-immune cells, and/or combinations thereof. Immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as CD4+ T cells, CD8+ T cells, B cells, NK cells, NK-T cells, and/or combinations thereof. In various embodiments, the T cells are Th1 cells, Th2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, hepatocytes, liver sinusoidal endothelial cells (LSECs), and/or Kupffer cells.

### Combination Therapy

Concurrent administration of two therapeutic agents does not require that the agents be administered at the same time or by the same route, as long as there is an overlap in the time period during which the agents are exerting their therapeutic effect. Prior, simultaneous or sequential administration is contemplated, as is administration on different days or weeks.

It is contemplated the TIMP-PBC and PBC therapeutic may be given concomitantly, or simultaneously, in the same formulation or separate formulation. It is further contemplated that the TIMP-PBC and PBC therapeutic are administered in a separate formulation and administered concomitantly, with concomitantly referring to agents given within 30 minutes to 12 hours of each other.

In another aspect, a TIMP-PBC and PBC therapeutic is administered prior to administration of the other composition. Prior administration refers to administration of TIMP-PBC and PBC therapeutic within the range of one week prior to treatment with the other therapy, up to 30 minutes before administration of the other therapy.

In another aspect, a TIMP-PBC and PBC therapeutic is administered subsequent to administration of the other composition. Subsequent administration refers to administration of TIMP-PBC and PBC therapeutic within the range of one week after treatment with the other therapy, up to 30 minutes after administration of the other therapy.

It is further contemplated that other adjunct or alternative therapies may be administered, where appropriate.

In various embodiments, TIMP-PBC is administered alone or in combination with a PBC therapeutic. In various embodiments, the therapeutic is administered prior to, concomitantly, or simultaneously, with or subsequent to the administration of TIMP-PBC.

In various embodiments, the therapeutic is selected from the group consisting of a steroid, corticosteroid, nonsteroidal immunosuppressive agent, immunomodulatory agent, monoclonal antibody, cytokine and chemokine targeting therapy, JAK inhibitor, chimeric antigen receptor (CAR) T-cell therapy, regulatory T-cell (Treg) therapy, B-cell targeting therapy, antiviral drugs, and surgical treatment. In various embodiments, the therapeutic is selected from the group consisting of ursodeoxycholic acid (UDCA), obeticholic acid (OCA), fibrates, peroxisome proliferator-activated receptor δ (pparδ) agonist, ileal bile acid transporter (IBAT) inhibitor, ustekinumab, tauroursodeoxycholic acid, 6α-ethyl chenodeoxycholic acid, setanaxib, moexipril, abatacept, fibroblast growth factor, statin, colchicone, CD20 targeting therapy, CD19 targeting therapy, CD40/CD40L targeting therapy, CD80/CD86 targeting therapy, B cell targeting factor (BAFF) targeting therapy, B cell maturation antigen (BCMA) targeting therapy, anti-IL6 therapy, anti-IFN therapy, amifampridine, cyclosporine, cyclophosphamide, batoclimab, inebilizumab, nipocalimab, pozelimab, rituximab, satralizumab, seldelapar, pentoxifylline, tocilizumab, tofacitinib, tolebrutininb, avorstatin, fenofibrate, bezafibrate, linerixibat, methotrexate, liver transplant, beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, and hydrocortisone.

In various embodiments, the therapeutic is administered 1, 2, 3, 4, 5, 6, or 7 days prior to administration of TIMP-PBC. In various embodiments, the therapeutic is administered 1, 2, 3, or 4 weeks prior to administration of TIMP-PBC. In various embodiments, the therapeutic is administered 1, 2, 3, 4, 5, 6, 7, 8, 9, 10, 11, or 12 months prior to administration of TIMP-PBC. In various embodiments, the therapeutic is administered 1, 2, 3, 4, 5, 6, 7, 8, 9, 10, 11, or 12 years prior to administration of TIMP-PBC. In various embodiments, the therapeutic is administered 1, 2, 3, 4, 5, 6, or 7 days subsequent to administration of TIMP-PBC. In various embodiments, the therapeutic is administered 1, 2, 3, or 4 weeks subsequent to administration of TIMP-PBC. In various embodiments, the therapeutic is administered 1, 2, 3, 4, 5, 6, 7, 8, 9, 10, 11, or 12 months subsequent to administration of TIMP-PBC. In various embodiments, the therapeutic is administered 1, 2, 3, 4, 5, 6, 7, 8, 9, 10, 11, or 12 years subsequent to administration of TIMP-PBC.

### Pharmaceutical Formulations

Pharmaceutical compositions of the present disclosure containing the TIMP-PBC described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carbox-yvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant, and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the inhibitors are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP-PBC described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a TIMP alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

In a further embodiment, the disclosure provides an article of manufacture, or unit dose form, comprising: (a) a composition of matter comprising TIMP-PBC as described herein; (b) a container containing said composition; and (c) a label affixed to said container, or a package insert included in said container referring to the use of said TIMP-PBC in the treatment of PBC as described herein.

Additional aspects and details of the disclosure will be apparent from the following examples, which are intended to be illustrative rather than limiting.

### EXAMPLES

### Example 1: TIMP-PBC Inhibits Antigen-Specific T Cell Responses in Animal Models

The efficacy of TIMP-PBC (CNP-104) encapsulating PBC antigen PDC-E2₁₅₅₋₁₈₅ (KVGEKLSEGDLLAEIETDKATIGFEVQEEGY) (SEQ ID NO: 1) at inducing T cell tolerance was examined in a therapeutic model of PBC.

In rodents, a PBC model can be induced with the chemical xenobiotic 2OA BSA which induces PBC-like cholangitis in C57BL/6 mice. In the PBC model, mice develop PDC-E2 specific anti-mitochondrial antibodies (AMA) in serum as well as infiltration of pathogenic T cells in liver bile ducts, mimicking human disease. Addition of poly(I:C) treatment to this model enhances CD8 T cell infiltration and results in liver fibrosis, a hallmark of human PBC.

On the day of experiment (Day 0), Naïve ˜six-week-old C57BL/6 mice were primed with 2OA-BSA by intraperitoneal (i.p) injection. 2OA-BSA i.p priming was repeated with Incomplete Freund's Adjuvant (IFA) on Day 14.

On Day 0 and Day 2, mice were injected i.p. with 100 ng of pertussis toxin (PTX). On Day 0 and Day 14, mice were injected subcutaneously (s.c.) with 5 mg/kg poly (I:C). On Day 7, mice were primed with PDC-E2₁₅₅₋₁₈₅/CFA emulsion. On Day 7 and Day 14, mice were treated with CNP-104 (2.5 mg dose/mouse or ˜10 mg/kg HED) or control nanoparticles.

On Day 22, mice in each treatment group (N=5 per group) were sacrificed and their spleens were harvested to assess antigen specific T cell responses and serum anti-mitochondrial antibody (AMA) titers.

Antigen specific T cell response from splenocytes: 5×10⁵ total splenocytes were plated in triplicate wells in 96-well flat-bottomed plates with complete RPMI medium with anti-CD3 antibody (0.5 μg/mL) and anti-CD28 (1 μg/mL) (positive control), or T cell specific PDC-E2₁₅₅₋₁₈₅ (titrated to 1.5 μg/mL, 15 μg/mL, 75 μg/mL).

Cells were collected at 72-hours after culture setup and the level of cell proliferation was assayed via WST-1 cell proliferation reagent (Roche, Cat #11644807001) according to manufacturer's protocol.

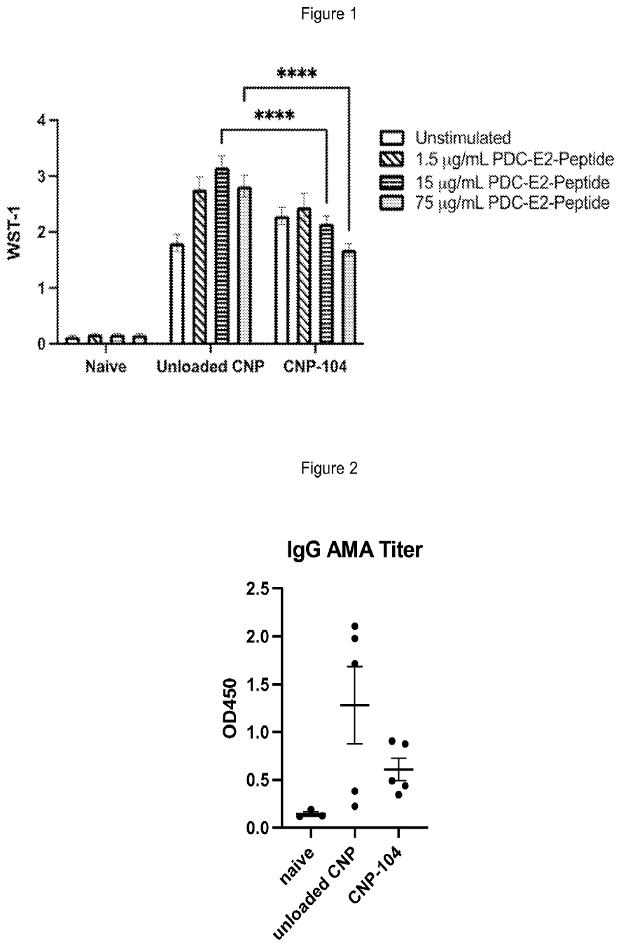

As shown in FIG. 1, CNP-104 treatment significantly inhibited PDC-E2₁₅₅₋₁₈₅ T cell response when compared to the control unloaded nanoparticle treatment (p<0.0001).

Anti-mitochondrial antibody titers from blood: Whole blood was collected from mice via retro-orbital route and placed in serum collection tubes. Blood was allowed to clot at room temperature for 2 hours. Serum was collected by centrifugation (3,000 g for 5 minutes at 4° C. Levels of AMAs were assayed using the QUANTA Lite® M2 EP (MIT3) ELISA kit using the PDC-E2 coated microwell/strips according to manufacturer's protocol. Mouse serum samples were added in duplicate to wells (100 μL sample/well). The plate was incubated at room temperature for 1 hour followed by washing with 1×PBS+0.05% Tween-20 four times.

IgG AMAs were detected by addition of anti-mouse IgG HRP secondary antibody (1:4000 dilution in 1×PBS+5% BSA) to each well followed by incubation for 1 hour at room temperature.

The plate was washed four times with 1×PBS+0.05% Tween-20. 100 μL of TMP One Component HRP Microwell Substrate was added to each well and developed at room temperature until sufficient color development occurred. 100 μL Stop Reagent was added to stop the reaction and the plate was read at 450 nm using an ELISA plate reader.

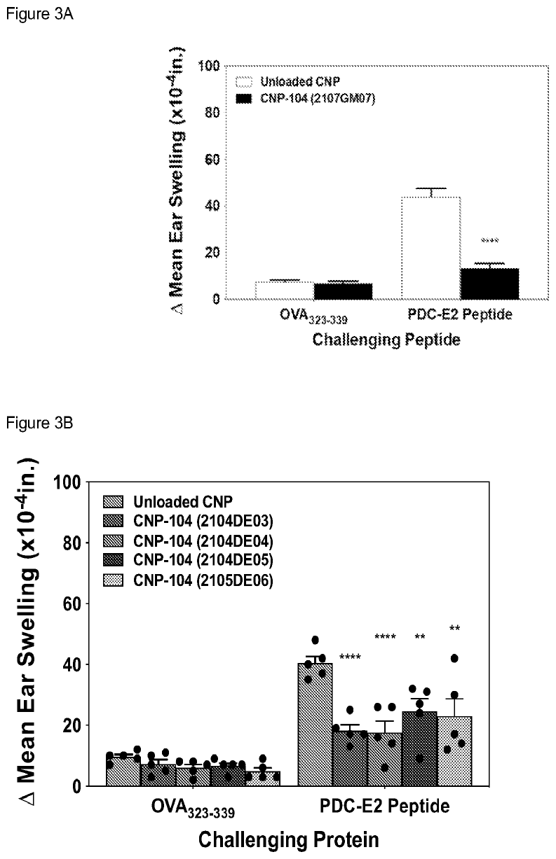

The optical density readings for all tested samples were recorded. As shown in FIG. 2, treatment with CNP-104 led to a reduction in serum IgG AMA titers when compared to control unloaded nanoparticles.

Delayed-Type Hypersensitivity: C57BL/6 mice were primed with 200 μg (100 μL injection volume) of PDC-E2-Peptide emulsified in CFA on Day 0. On Day 0 and Day 7 post priming, mice were treated with CNP 104 lots representative of the GLP manufacturing process administered via IV injection at a dose of 2.5 mg dose/mouse (10 mg/kg HED). On Day 14 post priming, the mice were challenged intradermally with 10 μg PDC-E2-Peptide (right ear, 10 μL injection volume) or Ovalbumin (left ear, 10 μL injection volume). The pinna thickness of each ear was measured immediately post-elicitation with PDC-E2-Peptide and OVA as well as 24 hours post elicitation. The change in pinna thickness (ΔT) for both ears of each mouse was calculated to assess the DTH response. CNP-104 significantly inhibited the DTH response compared to unloaded control particles at 2.5 mg/dose (10 mg/kg HED).

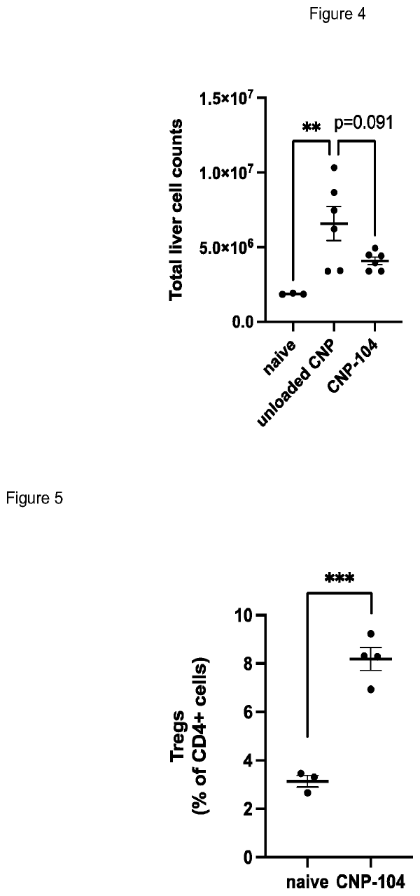

In a follow-up DTH experiment using CNP-104 representative of the GMP manufacturing process, CNP-104 significantly inhibited the DTH response compared to unloaded control particles at 2.5 mg/dose (10 mg/kg HED) (FIGS. 3A, 3B).

### Example 2: TIMP-PBC Inhibits Leukocyte Infiltration in Liver in the PBC Animal Model

The efficacy of TIMP-PBC (CNP-104) encapsulating PBC antigen PDC-E2₁₅₅₋₁₈₅ at inhibiting leukocyte infiltration in the liver was examined in a therapeutic model of PBC.

On the day of experiment (Day 0), Naïve ˜six-week-old C57BL/6 mice were primed with 2OA-BSA by intraperitoneal injection (i.p). 2OA-BSA i.p priming was repeated with Incomplete Freund's Adjuvant (IFA) on Day 14.

On Day 0 and Day 2, mice were injected i.p. with 100 ng of pertussis toxin (PTX). On Day 0 and Day 14, mice were injected subcutaneously (s.c.) with 5 mg/kg poly (I:C). On Day 7, mice were primed with PDC-E2₁₅₅₋₁₈₅/CFA emulsion. On Day 7 and Day 14, mice were treated with CNP-104 or control nanoparticles (N=6 per group).

A separate group of animals (N=3) was left unprimed and served as the Naïve control.

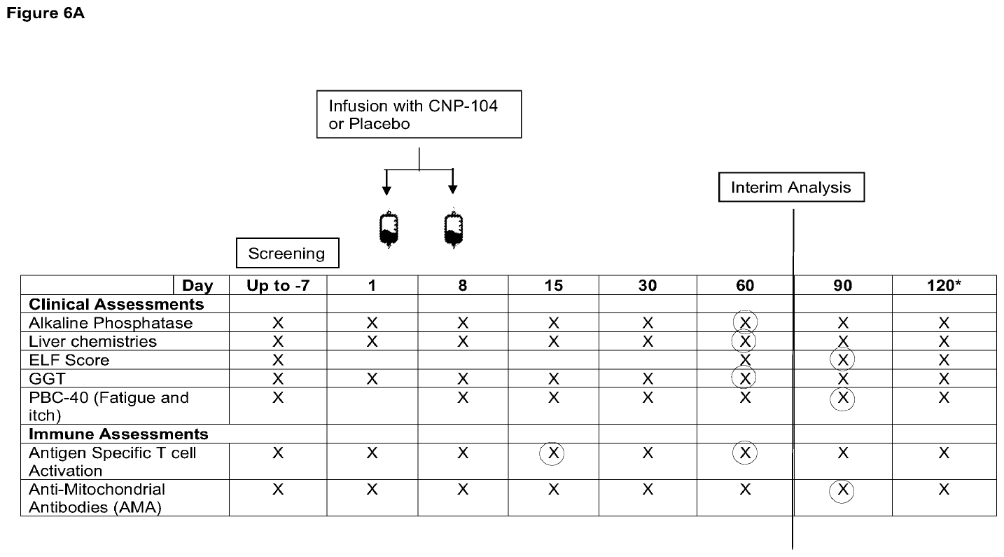

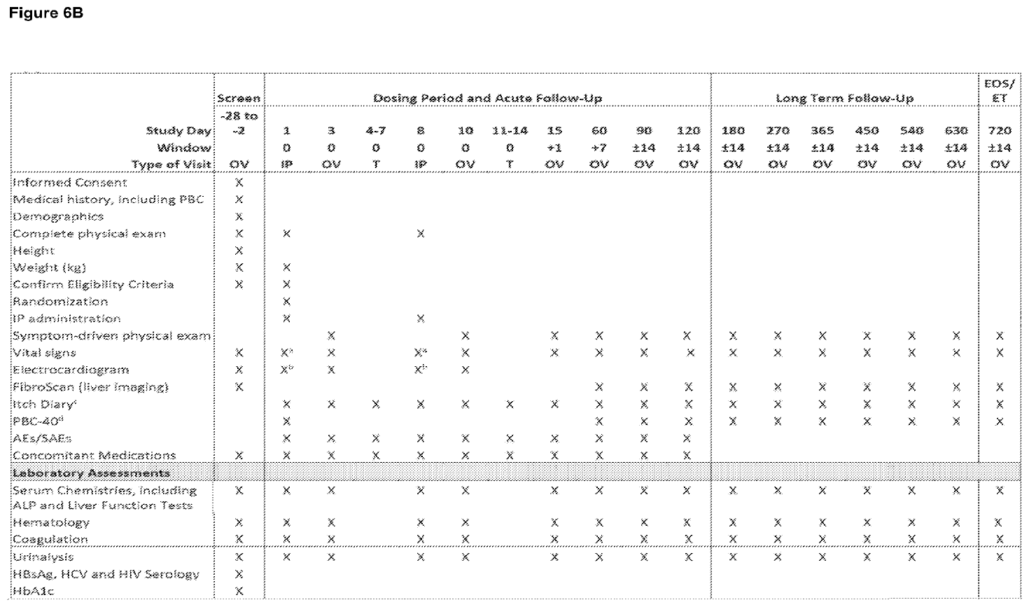

On Day 55, animals were sacrificed, and livers were harvested. Livers were processed to isolate leukocytes using gradient separation and the levels of leukocyte infiltrate in each group was assessed by flow cytometry by gating on live CD45+ cells. As shown in FIG. 4, treatment with CNP-104 led to a reduction in levels of leukocyte infiltrate in the liver when compared to control nanoparticle treatment. As shown in FIG. 5, treatment with CNP-104 led to a increase in Tregs (CD4⁺CD25⁺Foxp3⁺) in the liver compared to naïve untreated mice.

### Example 3: Phase 2a Trial of TIMP-PBC in Patients with PBC

A product specific repeat-dose dose range finding, and pivotal GLP toxicology studies were initially performed with CNP-104 (Study CNP-104-2.001 and Study CNP-104-2.002). These studies encompass and support the dose levels, administration frequency, and route of administration intended for the CNP-104 Phase 2a study.

A non-GLP repeat dose study was performed with CNP-104 being administered by intravenous infusion (IV) on Days 1, 8, and 15 at dose levels of 0 mg/kg, 25 mg/kg, 75 mg/kg, and 125 mg/kg (Study CNP-104-2.001). No drug-related effects were noted on body weight gain, food consumption, ophthalmology exams, physical exams, clinical observations, functional observational battery, body temperature and serum cytokine levels. Transient non-adverse clinical pathology observations quickly resolved. None of these findings were considered to be adverse. The highest dose administered intravenously of 125 mg/kg used in this study (administered on Days 1, 8 and 15) had no observed adverse effect and was therefore determined to be the NOAEL.

A pivotal GLP repeat dose study was performed with CNP-104 being administered by intravenous infusion (IV) on Days 1, 8, and 15 at dose levels of 0 mg/kg, 25 mg/kg, 75 mg/kg, and 125 mg/kg (Study CNP-104-2.002). No drug-related effects were noted on clinical observations or changes in body weight, food consumption, body temperature, or gross necropsy findings. Increases in cytokine parameters IL-5, IL-1β, IL-6 and IFN-γ in a few female rats were observed but were not considered to be adverse. The highest dose administered intravenously of 125 mg/kg used in this study (administered on Days 1, 8 and 15) had no observed adverse effect and was therefore determined to be the NOAEL.

A Phase 2a, double-blind, placebo-controlled study to evaluate the safety, tolerability, pharmacodynamics, and efficacy of CNP-104 in subjects ages 22-72 with Primary Biliary Cholangitis who are unresponsive to UDCA and/or OCA will be carried out.

CNP-104 consists of PLGA nanoparticles encapsulating PDC-E2₁₅₅₋₁₈₅ peptide. CNP-104 particles have an average diameter of approximately 400-800 nm and a negative zeta potential of between −30 mV and −60 mV. CNP-104 is supplied as a lyophilized formulation. CNP-104 is reconstituted in sterile water for injection and diluted in sterile saline (0.9% sodium chloride, USP) prior to administration.

Subjects ages 22-72 and 18-75 with primary biliary cholangitis will be screened up to 14 days prior to enrollment into the study. If subjects are currently on a standard of care therapy, they will remain on their current standard of care therapy during the course of the study at the discretion of the Investigator regardless of treatment group (CNP-104 or Placebo).

Screening will be completed to assess eligibility, obtain vital signs, collect laboratory samples and pharmacodynamic (PD) measurements, receive a fibroscan for liver fibrosis, and to collect a liver core biopsy for inflammation/fibrosis assessment.

Subjects will complete an initial PBC-40 assessment and begin an Itch Diary, a questionnaire and scoring system to be completed by the patient every morning and evening for the first 120 days of the study and then weekly through the duration of the study. PBC-40 is completed monthly from study day 1 through end of study. Subjects who meet all inclusion and no exclusion criteria after completing the screening visit will be enrolled in the study.

Prior to administration of study drug on Day 1, subjects will have a second set of liver laboratory samples collected. The results from these tests will be averaged with those obtained at the screening visit to provide a patient specific baseline ALT, total and direct bilirubin, and ALP values for monitoring of liver safety and detection of Drug Induced Liver Injury (DILI) throughout the course of the study.

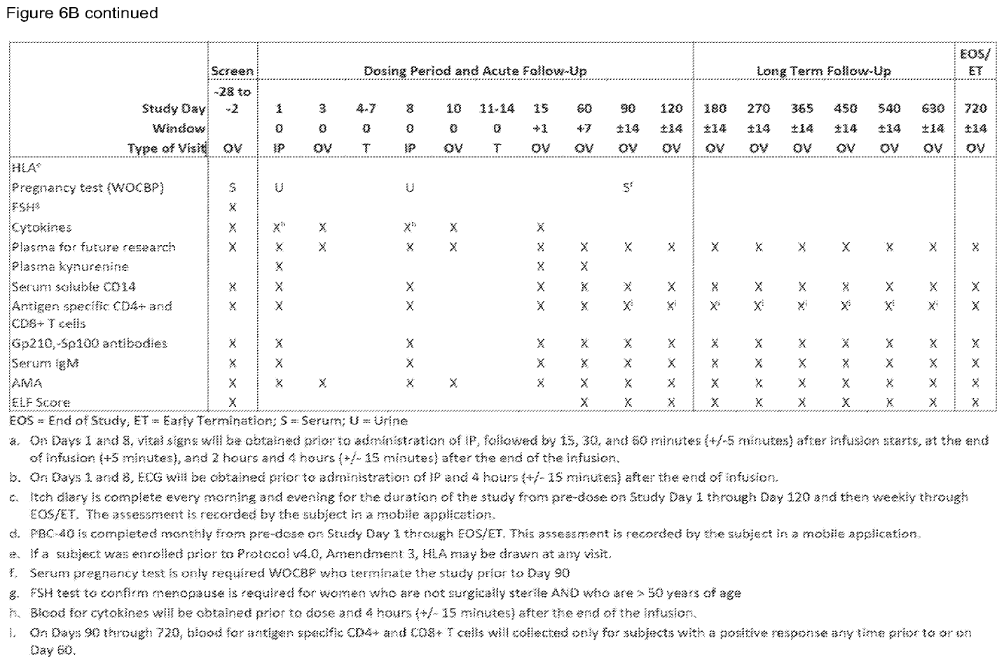

Subjects will then be randomized on Day 1 into the dose level Cohort open at the time in a 1:1 ratio to receive two separate administrations of intravenous CNP-104 or Placebo on Day 1 and Day 8 (FIG. 6). Study drug will be administered by IV infusion (3.25 mg/ml) over approximately 3-4 hours using a graduated rate of infusion as follows:
20 mL/hr for first the first 15 min, then
40 mL/hr, for the next 15 min, then
80 mL/hr for the remainder of the infusion.

Subjects will undergo medical observation in the clinic for acute AEs for 4 hours following infusion on Day 1 and Day 8. Subjects will be discharged after 4 hours if all scheduled assessments for the visit day have been completed, if vital signs (sitting or supine blood pressure, heart rate, and body temperature) measured 4 hours post-infusion within expected ranges for the subject, and if no other health concerns are noted by the Investigator.

Subjects will return for an office visit 2 days after each infusion (Day 3 and 10) for collection of safety labs, review of medications, and assessment of AEs and will be followed daily through telephone visits between infusions (Days 4-7 and 11-14) to assess and document any AEs and medication changes.

In the post-dosing period, subjects will return to the clinic for immune safety labs, PD measurements, PBC-40 assessment, assessment of AEs and medication changes (Days 15, 60, 90, 120, 180, 270, 365, 450, 540, 630, 700, and/or 730), fibroscan (Day 60, 90, 120, 180, 270, 365, 450, 540, 630, 700, and/or 730), and core liver biopsy (Day 60) (FIG. 6). Subjects will continue to complete the twice daily itch diaries throughout the study period up to Day 730. Subjects will return to the clinic for the end of study visit on Day 730 for collection of safety labs, PD measurements, PBC-40 assessment, fibroscan, and final assessment of AEs and medication changes.

The study is planned to enroll 2 cohorts at two dose levels. Subjects will be randomized in a 1:1 ratio to receive either CNP-104 or Placebo (0.9% sodium chloride injection, USP) as a 200 mL intravenous infusion on Day 1 and Day 8. The planned dose levels are as follows:
1. Cohort 1:4 mg/kg (N=6 subjects randomized 1:1)
2. Cohort 2:8 mg/kg (N=up to 34 subjects randomized 1:1)

Dosing of subjects within a given dose Cohort will be separated by at least 48 hours. After all subjects in a dose Cohort have completed the Day 15 office visit (7 days post-second dose), the DMC will be convened to review all available safety data and determine whether it is acceptable to proceed to the next dose Cohort, if an expansion of the Cohort is warranted, or if any other clinical recommendations should made.

Criteria for inclusion:
Subjects who are willing and able to provide Institutional Review Board (IRB) approved written informed consent and privacy language as per national regulations.
Men and non-pregnant, non-breast-feeding women, ages 22-27, or 18-75 years inclusive.
Subjects with a confirmed diagnosis of Primary Biliary Cholangitis based upon at least two of the following:
   Positive AMA titer (e.g., >1:40) or, if AMA negative or in low titer (<1:40), positive PBC-specific antibodies (anti-GP210 and/or anti-SP100 and/or antibodies against the major M2 components [PDC-E2, 2-oxo-glutaric acid dehydrogenase complex
   Alkaline phosphatase >1.5×ULN (Upper limit of normal) for at least 6 months
   Liver biopsy findings consistent with PBC
Subjects with a Class A Child-Pugh score.
Subjects who are unresponsive to UDCA and/or OCA after 6 months of treatment at a stable dose as measured by ALP >1.5×ULN or who are intolerance to UDCA and/or OCA
Subjects with ALP >1.5×ULN.
Subjects with AST and ALT≤10×ULN.
Subjects with positive antigen specific CD8+ T cells.
Men and women subjects of non-childbearing potential or women of child-bearing potential who have agreed not to pregnant during the study, have a negative pregnancy test at both screening visits and prior to each dose and agree to practice a highly effective method of contraception that may include, but is not limited to, abstinence, sex only with persons of the same sex, monogamous relationship with vasectomized partner, vasectomy, hysterectomy, bilateral tubal ligation, licensed hormonal methods, intrauterine device (IUD) starting at initial screening and continuing throughout the entire study to Day 90.
Female subjects who agree to not breastfeed starting at initial screening and throughout the entire study to Day 120.
Female subjects who agree to not donate ova starting at initial screening and throughout the entire study to Day 90.
Male subjects who agree to not donate sperm starting at screening and throughout the entire study to Day 90.

Criteria for exclusion:
Subjects with greater than early Stage 3 Primary Biliary Cholangitis.
Subjects with Class B or Class C Child-Pugh score.
Subjects with concomitant liver disease including viral hepatitis, PSC, alcoholic liver disease, Wilson's diseases, hemochromatosis, or Gilbert's syndrome.
Subjects who have previously undergone liver transplantation.
Subjects with decompensated liver disease as defined by the presence or history of any of the following:
   MELD score >15
   Hepatic encephalopathy
   Ascites
   Hepatorenal syndrome or serum creatinine >2 mg/dL
   Total bilirubin >3.0 mg/dL
   INR >1.8 unless on anticoagulation such as Coumadin
   History of variceal hemorrhage
Subjects with a history of cerebrovascular accident in the past 12 months.
Subjects with a history of myocardial infarction, as defined by any of the following criteria:
   Development of pathological Q waves with or without symptoms
   Imaging evidence of a region of loss of viable myocardium that is thinned and fails to contract, in the absence of a non-ischemic cause
   Pathological findings of a healed or healing myocardial
Subjects with chronic kidney disease, as defined by Glomerular Filtration Rate (GFR) <60 mL/min/1.73 m² for at least 3 months where GFR is calculated based on the CKD-EPI equation:

EGFR=142×min(Scr/κ,1)^{α}×max(Scr/κ,1)^{-1.200}×0.9938^{Age}×1.018[if female], or:

GFR=141×min(Scr/κ,1)α×max(Scr/κ,1)−1.209×0.993Age×1.018[if female]×1.159[if black] where:
Scr is serum creatinine in mg/dL
κ is 0.7 for females and 0.9 for males
α is −0.329 for females and −0.411 for males
Min indicates the minimum of Scr/κ or 1
Max indicates the maximum of Scr/κ or 1
Subjects with uncontrolled diabetes, as defined by HbA1c>7%.
Subjects who have used biologic agents including anti-cell and anti-cytokine therapies within 12 months of Day 0 or prior to screening.
Subjects with history of tuberculosis or positive PPD skin test.
Subjects who have received administration of any live vaccine (other than intranasal influenza vaccine) within 28 days or subunit vaccine within 14 days prior to screening 1 or are planning to receive any vaccination before day 90.
Subjects who have received any COVID-19 vaccine (either first or second dose) within 14 days prior to Screening. Subjects who have received the first dose of any COVID-19 vaccine may not screen for the study until 14 days following their second dose of the vaccine if applicable.
Subjects who have used systemic steroids within 3 months prior to screening.
Subjects with laboratory test results at screening or prior to study dosing that are outside the normal limits and considered by the investigator to be clinically significant. This criterion does not apply to liver function tests. Additionally, clinically significant laboratory test results at screening that are related to the condition (PBC) are acceptable.
Subjects with positive test results for hepatitis B surface antigen (HBsAg), hepatitis C virus (HCV) antibody, or human immunodeficiency virus (HIV) antigen/antibody as determined at screening.
Subjects with a history of or currently active immune disorders other than PBC (including autoimmune disease) unless the condition, after discussing with the Medical Monitor, has been deemed to be acceptable for the subject's participation in this study
Subjects with a history of or current active diseases requiring immunosuppressive drugs (including azathioprine, prednisone, prednisolone, budesonide, cyclosporine, tacrolimus, methotrexate, or mycophenolate mofetil) unless the condition, after discussion with the Medical Monitor, has been deemed to be acceptable for the subject's participation in this study
Subjects with a clinical history of significant cardiovascular disease as determined by the Investigator.
Subjects with a complication or medical history of malignancy within the past 5 years which, in the Investigator's option, makes the subject unsuitable for study participation.
Subjects who, in the Investigator's opinion, will be unable to adhere to study procedures.
Subjects who have received an investigational therapy other than CNP-104 within 28 days or 5 half-lives, whichever is longer, prior to screening.
Subjects with any condition which, in the investigator's opinion, makes the subject unsuitable for study participation.
Known sensitivity to any components of CNP-104.

Primary objectives of the study include to assess the safety and tolerability of CNP-104, and to assess the change in Serum Alkaline Phosphatase (ALP) levels among patients treated with CNP-104 or placebo.

Secondary endpoints include assessing change in levels of bilirubin, albumin, ALT, AST, IFN-γ, GGT, Gp210, Sp100, soluble CD14, serum IgM, serum kynurenine, inflammation in liver, liver fibrosis in patients receiving CNP-104 compared to placebo, and to assess the change in T cell infiltrate in the liver by core biopsy among patients treated with CNP-104 or placebo. Assessment of patients' ELF score, Worst Daily Itch Score and PBC-40 scale are also assessed.

Subjects randomized to test product will receive either 4 mg/kg or 8 mg/kg (up to a maximum dose of 650 mg per day) of CNP-104 as a 200 mL intravenous infusion on Day 1 and Day 8.

Throughout the course of the study, subjects will be closely monitored for potential signs of both hepatocellular and cholestatic drug induced liver injury (DILI). Monitoring of liver tests is divided into three categories: Subjects with normal baseline (subject specific mean baseline) ALT values with values with Hepatocellular DILI signals, subjects with elevated baseline (subject specific mean baseline) ALT values with Hepatocellular DILI signals, and subjects with Cholestatic DILI signals. All liver test values will be compared to the subject specific mean baseline value (“baseline”) from two tests obtained prior to the exposure to the suspected drug.

For subjects with normal baseline ALT values, the following monitoring will occur for Hepatocellular DILI signals:

| TREATMENT- | | LIVER- | |
|---|---|---|---|
| EMERGENT | | RELATED | |
| ALT | BILIRUBIN | SYMPTOMS | ACTION |
| ALT ≥5 × ULN | Normal | None | Blood tests will be repeated in 2-5 |
| | | | days. |
| | | | Subject will be followed for symptoms. |
| ALT ≥8 × ULN | Normal or | None or | Interrupt study drug. |
| | elevated | present | Blood tests will be repeated within 2-5 |
| | | | days. |
| | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |
| ALT ≥3 × ULN | Total | None or | Interrupt study drug. |
| | bilirubin ≥2 × | present | Blood tests will be repeated within 2-5 |
| | baseline | | days. |
| | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |
| ALT ≥ 5 × ULN | Normal or | Present | Study drug will be interrupted. |
| | elevated | | Blood tests will be repeated within 2-5 |
| | | | days. |
| | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |

For subjects with elevated baseline ALT values, the following monitoring will occur for Hepatocellular DILI signals:

| TREATMENT- | | LIVER- | |
|---|---|---|---|
| EMERGENT | | RELATED | |
| ALT | BILIRUBIN | SYMPTOMS | ACTION |
| ALT ≥3 × baseline | Normal | None | Blood tests will be repeated in 2-5 days. |
| or ≥300 U/L | | | Subject will be followed for symptoms. |
| (whichever | | | |
| occurs first) | | | |
| ALT ≥5 × baseline | Normal or | None or | Interrupt study drug. |
| or ≥500 U/L | elevated | present | Blood tests will be repeated within 2-5 |
| (whichever | | | days. |
| occurs first) | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |
| ALT ≥2 × baseline | Total | None or | Interrupt study drug. |
| or ≥300 U/L | bilirubin ≥2 × | present | Blood tests will be repeated within 2-5 |
| (whichever | baseline | | days. |
| occurs first) | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |
| ALT ≥2 × baseline | Normal or | Present | Study drug will be interrupted. |
| or ≥300 U/L | elevated | | Blood tests will be repeated within 2-5 |
| (whichever | | | days. |
| occurs first) | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |

The following monitoring will occur for Cholestatic DILI signals:

| | | LIVER- | |
|---|---|---|---|
| TREATMENT- | | RELATED | |
| EMERGENT ALT | BILIRUBIN | SYMPTOMS | ACTION |
| ALP ≥2 × baseline | Normal | None | Blood tests will be repeated in 7-10 |
| without alternative | | | days. |
| explanation | | | Subject will be followed for symptoms. |
| ALP ≥2 × baseline | Total | None or | Interrupt study drug. |
| without alternative | bilirubin ≥2 × | present | Blood tests will be repeated within 7-10 |
| explanation | baseline | | days. |
| | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |
| ALP ≥2 × baseline | Normal or | Present | Interrupt study drug. |
| without alternative | elevated | | Blood tests will be repeated within 7-10 |
| explanation | | | days. |
| | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |
| ALP ≥3 × baseline | Normal or | None or | Study drug will be interrupted. |
| without alternative | elevated | present | Blood tests will be repeated within 2-5 |
| explanation | | | days. |
| | | | Close monitoring and workup for |
| | | | competing etiologies will be initiated. |
| | | | Study drug can be restarted only if |
| | | | another etiology is identified and liver |
| | | | enzymes return to baseline. |
| | | | Drug cannot be restarted if hepatic |
| | | | decompensation occurred. |

Blood tests (ALT, total and direct bilirubin, and ALP) should be repeated within 2-5 days if hepatocellular DILI is suspected, and within 7-10 days if cholestatic DILI is suspected to confirm the reproducibility of the initial value as well as the directionality of change from the initial value.

Safety assessments will include:
Complete physical examination at Screen and Days 1 and 8, and symptom driven physical exams on Days 3, 10, 15, 60, 90, 120, 180, 270, 365, 450, 540, 630, 700, and 720;
Vital signs at Screen and Days 1, 3, 8, 10, 15, 60, 90, 120, 180, 270, 365, 450, 540, 630, 700, and 720; optionally, followed by symptom driven physical exams on Days 15, 60, 90, 120, 180, 270, 365, 450, 540, 630, 700, and 720;
12-lead electrocardiograms (ECGs) at Screen and Days 1, 3, 8, and 10;
AEs at Screen followed by Days 1, 3, 4-7 (remote), 8, 10, 11-14 (remote), 15, 60, 90, 120, 180, 270, 365, 450, 540, 630, 700, and 720;
Safety laboratory tests (chemistry, hematology, coagulation and urinalysis) at Screen followed by Days 1, 3, 8, 10, 15, 60, 90, 120, 180, 270, 365, 450, 540, 630, 700, and 720;
Cytokine (IL-1β, TNF-α, IL-6, MCP-1, MIP-1α, IFN-γ, IL-4, IL-10) profiling at Screen followed by Days 1, 3, 8, 10 and 15;
Anti-mitochondrial antibodies at Screen followed by Days 1, 3, 8, 10, 15, 60, 90, 120, 180, 270, 365, 450, 540, 630, 700, and 720.

Laboratory/PD assessments will include:
T cell infiltrate (from liver core biopsy) at Screen followed by Day 60;
Antigen Specific CD4+ and CD8+ T cells at Screen followed by Days 1, 8, 15, 60, and 90;
Serum Alkaline Phosphatase (ALP) at Screen followed by Days 1, 8, 15, 60, 90, 120, 180, 270, 365, 450, 540, 630, 700, and 720;
Liver chemistries (Bilirubin, Albumin, ALT, AST) at Screen followed by Days 1, 8, 15, 60, 90, 120, 180, 270, 365, 450, 540, 630, 700, and 720;
Antinuclear autoantibodies (Gp210 and Sp100) at Screen followed by Days 1, 8, 15, 60, 90, 120, 180, 270, 365, 450, 540, 630, 700, and 720;
Serum IgM at Screen followed by Days 1, 8, 15, 60, 90, 120, 180, 270, 365, 450, 540, 630, 700, and 720;
Inflammation/fibrosis (from liver core biopsy) at Screen followed by Day 60;
Serum soluble CD14 at Screen followed by Days 1, 8, 15, 60, 90, 120, 180, 270, 365, 450, 540, 630, 700, and 730;
Serum kynurenine at day 1 followed by Days 15, 60, optionally at days 1, 8, 90, 120, 180, 270, 365, 450, 540, 630, 700, and 720.

Clinical efficacy assessments will include:
Liver stiffness (fibroscan) at Screen followed by Days 60, 90, 120, 180, 270, 365, 450, 540, 630, 700, and 720;
ELF score at Screen followed by Days 60, 90, 120, 180, 270, 365, 450, 540, 630, 700, and 720;
PBC-40 assessment at day 1 followed by Days 8, 15, 60, 90, 120, 180, 270, 365, 450, 540, 630, 700, and 720.
Itch Diary at day 1 followed by every subsequent Study Day through Day 120 and then weekly through Day 730.

### Example 4: Patient Selection Criteria for CNP 104 Treatment

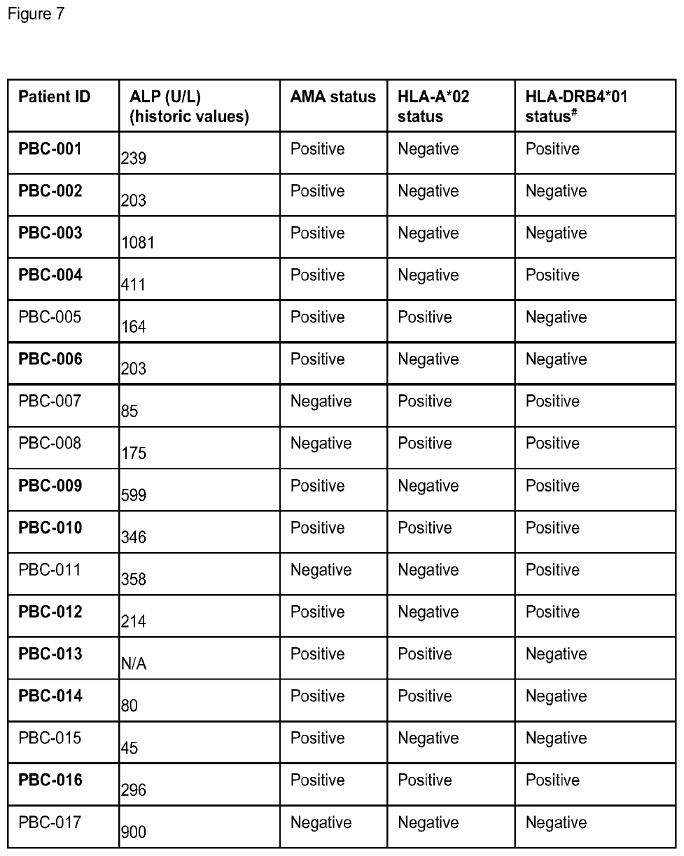

PBMCs were obtained from 17 PBC patients to determine if there was a correlation between AMA positivity, PDC-E2 restricted HLA positivity and ALP levels. 70% of patients in the study expressed an HLA restriction, either HLA-A*02 or HLA-DRB4*01 or both, for PDC-E2. Since CNP 104 encapsulates PDC-E2 subjects with HLA-A*02 or HLA-DRB4*01, status can be a criteria for inclusion in the CNP 104 clinical trial. 75% patients were positive for AMAs. Four patients out of 17 were negative for AMA but 3 of the 4 had positive HLA phenotypes for CNP-104. 70% of PBC patients had elevated ALP levels in blood. Selection criteria for CNP 104 treatment can be based on HLA haplotype restriction, AMA positivity, ALP levels. Selection criteria could identify patients likely to achieve efficacy with CNP 104. (FIG. 7). PBC patients whose disease is driven by autoreactivity to the PDC-E2 antigen are eligible for treatment with CNP 104 and can be identified based on HLA haplotype, anti-mitochondrial antibodies and levels of alkaline phosphatase in blood.

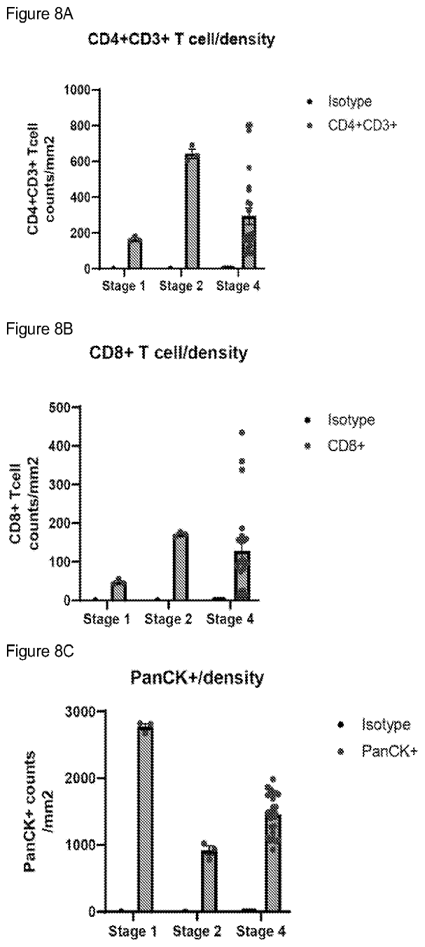

Liver biopsies from PBC patients were evaluated for presence of infiltrating T-cells as a potential criteria for inclusion in a clinical trial of CNP 104. Severity of liver damage was scored on a scale of 1-4 with 4 having most severe liver pathology. The cell counts for CD4+ T cells (FIG. 8A), CD8+ T cells (FIG. 8B) and biliary epithelial cells PanCK+ (FIG. 8C) were detected in stage 1, 3 and 4 PBC livers over isotype control. Selection criteria for CNP 104 treatment can be based on the presence of PDC-E2 specific CD4+ and/or CD8+ T cells in the blood or liver (FIG. 8).

It is understood that every embodiment of the disclosure described herein may optionally be combined with any one or more of the other embodiments described herein. Every patent literature and every non-patent literature cited herein are incorporated herein by reference in their entirety.

It is understood, therefore, that this invention is not limited to the particular embodiments disclosed, but is intended to cover all modifications which are within the spirit and scope of the invention as defined by the appended claims; the above description, and/or shown in the attached drawings. Consequently only such limitations as appear in the appended claims should be placed on the disclosure.

### REFERENCES

1. Lu M, Zhou Y, Haller I V, et al. Increasing Prevalence of Primary Biliary Cholangitis and Reduced Mortality With Treatment. Clin Gastroenterol Hepatol. 2018; 16(8):1342-1350.e1341.
2. Webb G J, Siminovitch K A, Hirschfield G M. The immunogenetics of primary biliary cirrhosis: A comprehensive review. J Autoimmun. 2015; 64:42-52.
3. Gershwin M E, Ansari A A, Mackay I R, et al. Primary biliary cirrhosis: an orchestrated immune response against epithelial cells. Immunol Rev. 2000; 174:210-225.
4. Jones D E J. Autoantigens in primary biliary cirrhosis. Journal of Clinical Pathology. 2000; 53 (11): 813-821.
5. Hu C J, Zhang F C, Li Y Z, Zhang X. Primary biliary cirrhosis: what do autoantibodies tell us? World J Gastroenterol. 2010; 16 (29): 3616-3629.
6. Bowlus C L, Kenney J T, Rice G, Navarro R. Primary Biliary Cholangitis: Medical and Specialty Pharmacy Management Update. J Manag Care Spec Pharm. 2016; 22 (10-a-s Suppl):S3-s15.
7. Aguilar M T, Chascsa D M. Update on Emerging Treatment Options for Primary Biliary Cholangitis. Hepat Med. 2020; 12:69-77.
8. Lindor K D, Bowlus C L, Boyer J, Levy C, Mayo M. Primary Biliary Cholangitis: 2018 Practice Guidance from the American Association for the Study of Liver Diseases. Hepatology. 2019; 69(1):394-419.
9. Hiramatsu K, Aoyama H, Zen Y, Aishima S, Kitagawa S, Nakanuma Y. Proposal of a new staging and grading system of the liver for primary biliary cirrhosis. Histopathology. 2006; 49(5):466-478.
10. Kakuda Y, Harada K, Sawada-Kitamura S, et al. Evaluation of a new histologic staging and grading system for primary biliary cirrhosis in comparison with classical systems. Hum Pathol. 20130; 44(6):1107-1117.

What is claimed is:

1. A method of treating primary biliary cholangitis (PBC) in a subject comprising administering to the subject tolerizing immune modifying particles encapsulating one or more PBC antigens (TIMP-PBC), wherein TIMP-PBC is administered at a dose level of between about 0.01 mg/kg and 12 mg/kg.

2. The method of claim 1, wherein the PBC antigen is pyruvate dehydrogenase complex E-2 (PDC-E2).

3. The method of claim 1 or 2, wherein the PBC antigen comprises PDC-E2 amino acid residues 155-185 (PBC₁₅₅₋₁₈₅) (SEQ ID NO: 1).

4. The method of any one of claims 1 to 3, wherein the PBC antigen is PBC₁₅₅₋₁₈₅ peptide (SEQ ID NO: 1).

5. The method of any one of the preceding claims, wherein the TIMP-PBC particles have an average diameter of between 100 nm and 1500 nm.

6. The method of any one of the preceding claims, wherein the TIMP-PBC particles have a negative zeta potential.

7. The method of any one of the preceding claims, wherein the particles have a negative zeta potential of between −30 mV and −100 mV.

8. The method of any one of the preceding claims, wherein TIMP-PBC is administered at a concentration of between about 0.005 mg/mL and about 50 mg/mL, optionally about 0.05 mg/ml, 0.1 mg/mL, 0.5 mg/mL, 1 mg/mL, 2 mg/mL, 3 mg/ml, 3.25 mg/ml, 3.5 mg/ml, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

9. The method of any one of the preceding claims, wherein TIMP-PBC is administered at a dose level of about 0.01 mg/kg, 0.05 mg/kg, 0.1 mg/kg, 0.25 mg/kg, 0.5 mg/kg, 1.0 mg/kg, 2.0 mg/kg, 4.0 mg/kg, 6 mg/kg, 8.0 mg/kg, 10 mg/kg, or 12 mg/kg.

10. The method of any one of the preceding claims, wherein TIMP-PBC is administered at a dose level of between about 1 mg and 800 mg.

11. The method of any one of the preceding claims, wherein TIMP-PBC is administered at a dose of about 1 mg, 2 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg.

12. The method of any one of the preceding claims, wherein TIMP-PBC is administered in a single dose or in multiple doses.

13. The method of any one of the preceding claims, wherein TIMP-PBC is administered once weekly, once every two weeks, once every three weeks, once every 4 weeks, once every two months, once every three months, once every 6 months, or once per year.

14. The method of any one of the preceding claims, wherein TIMP-PBC is administered in two doses one week apart.

15. The method of any one of the preceding claims, wherein TIMP-PBC is administered intravenously, subcutaneously, intramuscular, intraperitoneally, intranasally, or orally.

16. The method of any one of the preceding claims, wherein administering TIMP-PBC to a subject reduces or ameliorates one or more symptoms of PBC.

17. The method of claim 16, wherein the one or more symptoms of PBC are selected from the group consisting of liver inflammation, cirrhosis, cholestasis, liver dysfunction, liver failure, liver fibrosis, increased liver immune infiltrate, elevated bile acid levels, elevated liver enzyme levels, (ALT, ALP, AST, γ-glutamyl transpeptidase), elevated bilirubin levels, circulating anti-mitochondrial antibodies (AMAs), circulating anti-nuclear antibodies (ANAs), fatigue, itchy skin, pruritis, dry eyes and mouth, abdominal pain, splenomegaly, musculoskeletal pain, edema, fluid buildup, skin xanthomas, jaundice, hyperpigmentation, osteoporosis, high cholesterol, diarrhea, steatorrhea, hypothyroidism, and weight loss.

18. The method of any one of the preceding claims, wherein administering TIMP-PBC in a subject reduces duration and/or severity of an inflammatory immune response to PBC antigens.

19. The method of claim 18, wherein the inflammatory immune response is a T cell, B cell, or a myeloid cell response.

20. The method of claim 18 or 19, wherein the inflammatory immune response is assayed from one or more biological samples obtained from the subject.

21. The method of claim 19, wherein the biological sample is selected from the group consisting of whole-blood, peripheral blood, peripheral blood mononuclear cells (PBMCs), serum, plasma, urine, cerebrospinal fluid (CSF), stool, a tissue biopsy, and/or a bone-marrow biopsy.

22. The method of claim 21, wherein the tissue biopsy is a liver biopsy.

23. The method of claim 22, wherein the liver biopsy is a core liver biopsy.

24. The method of any one of the preceding claims, wherein administering TIMP-PBC in a subject reduces levels of activated antigen-specific T cells.

25. The method of claim 24, wherein administering TIMP-PBC in a subject reduces levels of activated antigen-specific CD4+ and/or CD8+ T cells.

26. The method of any one of the preceding claims, wherein administering TIMP-PBC in a subject reduces levels of T cell infiltrate in the liver.

27. The method of claim 26 wherein, treatment with TIMP-PBC decreases liver immune infiltrate by about 5%-100% or by about 2-100-fold.

28. The method of any one of the preceding claims, wherein administering TIMP-PBC in a subject reduces levels of anti-mitochondrial antibodies in blood.

29. The method of claim 28, wherein treatment with TIMP-PBC decreases the levels of anti-mitochondrial antibodies by about 5%-100% or by about 2-100-fold relative to the subject's baseline measurement and/or relative to a healthy subject.

30. The method of any one of the preceding claims, wherein administering TIMP-PBC in a subject reduces levels of anti-nuclear antibodies in blood.

31. The method of claim 30, wherein treatment with TIMP-PBC decreases the levels of anti-nuclear antibodies by about 5%-100% or by about 2-100-fold relative to the subject's baseline measurement and/or relative to a healthy subject.

32. The method of any one of the preceding claims, wherein administering TIMP-PBC in a subject reduces levels of IgM in blood.

33. The method of claim 32, wherein treatment with TIMP-PBC decreases the levels of IgM by about 5%-100% or by about 2-100-fold relative to the subject's baseline measurement and/or relative to a healthy subject.

34. The method of any one of the preceding claims, wherein administering TIMP-PBC in a subject reduces levels of anti-Gp210 antibody in blood.

35. The method of any one of the preceding claims, wherein administering TIMP-PBC in a subject reduces levels of anti-Sp100 antibody in blood.

36. The method of any one of the preceding claims, wherein administering TIMP-PBC in a subject reduces levels of kynurenine in blood.

37. The method of any one of the preceding claims, wherein administering TIMP-PBC in a subject reduces levels of soluble CD14 in blood.

38. The method of any one of claims 34-37, wherein treatment with TIMP-PBC decreases the levels of anti-Gp210 antibody, anti-Sp100 antibody, kynurenine and/or CD14 by about 5%-100% or by about 2-100-fold relative to the subject's baseline measurement and/or relative to a healthy subject.

39. The method of claim 16, wherein amelioration in one or more symptoms of PBC is determined using PBC-40 scale, Enhanced Liver Fibrosis (ELF) score, PBC-27 scale, GLOBE scale, UK-PBC scale, Mean Worst Daily Itch scale, 5-D itch scale, Visual Analogue Scale (VAS), Scheuer Staging, Nakanuma Staging, Fibrosis Score, Bile Duct Loss Score, FIB-4 Index, or APRI.

40. The method of any one of the preceding claims, wherein administering TIMP-PBC in a subject reduces liver fibrosis.

41. The method of claim 40, wherein liver fibrosis is assessed from a liver biopsy.

42. The method of claim 40, wherein liver fibrosis is assessed by imaging.

43. The method of claim 42, wherein liver fibrosis is assessed by FibroScan, ultrasonography, computed tomography (CT), magnetic resonance imaging (MRI), magnetic resonance elastography (MRA), ultrasound elastography, transient elastography, point shear wave elastography, or two-dimensional shear wave elastography.

44. The method of any one of claims 40-43, wherein treatment with TIMP-PBC decreases the liver fibrosis by about 5%-100% or by about 2-100-fold.

45. The method of any one of the preceding claims, wherein administering TIMP-PBC in a subject alters the coagulation profile.

46. The method of claim 45, wherein the coagulation profile consists of activated partial thromboplastin time [aPTT], prothrombin time [PT], international normalized ratio [INR]).

47. The method of any one of the preceding claims, wherein administering TIMP-PBC reduces the use of alternative therapies.

48. The method of claim 47, wherein the therapy is selected from the group consisting of a steroid, corticosteroid, nonsteroidal immunosuppressive agent, immunomodulatory agent, monoclonal antibody, cytokine and chemokine targeting therapy, JAK inhibitor, chimeric antigen receptor (CAR) T-cell therapy, regulatory T-cell (Treg) therapy, B-cell targeting therapy, antiviral drugs, synthetic bile acids, and surgical treatment.

49. The method of claim 47 or 48, wherein the therapy is ursodeoxycholic acid (UDCA), obeticholic acid (OCA), fibrates, peroxisome proliferator-activated receptor δ (pparδ) agonist, ileal bile acid transporter (IBAT) inhibitor, tauroursodeoxycholic acid, 6α-ethyl chenodeoxycholic acid, setanaxib, moexipril, abatacept, fibroblast growth factor, statin, colchicone, ustekinumab, CD20 targeting therapy, CD19 targeting therapy, CD40/CD40L targeting therapy, CD80/CD86 targeting therapy, B cell targeting factor (BAFF) targeting therapy, B cell maturation antigen (BCMA) targeting therapy, anti-IL6 therapy, anti-IFN therapy, amifampridine, cyclosporine, cyclophosphamide, batoclimab, inebilizumab, nipocalimab, pozelimab, rituximab, satralizumab, seldelapar, pentoxifylline, tocilizumab, tofacitinib, tolebrutininb, avorstatin, fenofibrate, bezafibrate, linerixibat, methotrexate, butyrate, palmitate, liver transplant, beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, or hydrocortisone.

50. The method of any one of claims 47-49, wherein administration of TIMP-PBC decreases use of alternative PBC therapies in the subject 1%-100%.

51. The method of any one of the preceding claims, wherein TIMP-PBC is administered alone or in combination with a PBC therapeutic.

52. The method of claim 51, wherein the therapeutic is selected from the group consisting of a steroid, corticosteroid, nonsteroidal immunosuppressive agent, immunomodulatory agent, monoclonal antibody, cytokine and chemokine targeting therapy, JAK inhibitor, chimeric antigen receptor (CAR) T-cell therapy, regulatory T-cell (Treg) therapy, B-cell targeting therapy, antiviral drugs, synthetic bile acids, and surgical treatment.

53. The methods of claim 51 or 52, wherein the therapeutic is selected from the group consisting of ursodeoxycholic acid (UDCA), obeticholic acid (OCA), fibrates, peroxisome proliferator-activated receptor δ (pparδ) agonist, ileal bile acid transporter (IBAT) inhibitor, ustekinumab, tauroursodeoxycholic acid, 6α-ethyl chenodeoxycholic acid, setanaxib, moexipril, abatacept, fibroblast growth factor, statin, colchicone, CD20 targeting therapy, CD19 targeting therapy, CD40/CD40L targeting therapy, CD80/CD86 targeting therapy, B cell targeting factor (BAFF) targeting therapy, B cell maturation antigen (BCMA) targeting therapy, anti-IL6 therapy, anti-IFN therapy, amifampridine, cyclosporine, cyclophosphamide, batoclimab, inebilizumab, nipocalimab, pozelimab, rituximab, satralizumab, seldelapar, pentoxifylline, tocilizumab, tofacitinib, tolebrutininb, avorstatin, fenofibrate, bezafibrate, linerixibat, methotrexate, butyrate, palmitate, liver transplant, beclomethasone, ciclesonide, fluticasone furoatr, mometasone, budenoside, fluticasone, triamcinolone, loteprednol, cortisone, prednisone, prednisolone, methylprednisolone, triamsinolone, dexamethasone, betamethasone, and hydrocortisone.

54. The method of any one of claims 51-53, wherein the therapeutic is administered prior to, concomitantly with or subsequent to the administration of TIMP-PBC.
